(19) United States
(12) Patent Application Publication
(10) Pub. No.: US 2025/0120619 A1
(43) Pub. Date: Apr. 17, 2025

(54) METHOD AND SYSTEM FOR GLYCEMIC PREDICTION AND DYNAMIC VISUALIZATION

(71) Applicant: Abbott Diabetes Care Inc., Alameda, CA (US)

(72) Inventors: Erwin Satrya BUDIMAN, Fremont, CA (US); Yue RUAN, Oxford (GB)

(21) Appl. No.: 18/913,958

(22) Filed: Oct. 11, 2024

Related U.S. Application Data
(60) Provisional application No. 63/589,477, filed on Oct. 11, 2023.

(51) Int. Cl. A61B 5/145; A61B 5/00; A63B 24/00

(52) U.S. Cl. CPC A61B 5/14532; A61B 5/0004; A61B 5/14503; A61B 5/7275; A61B 5/7435; A61B 5/7475; A63B 24/0062

(57) ABSTRACT

Disclosed herein are system, method, and computer program product embodiments for interconnecting a prediction visualization with user medical data for analyzing the impact of personal choices on future glucose levels. The prediction visualization is configured to generate predictions of glycemic impact based one or more inputs including choices involving diet and exercise and user medical data, such as the user's historical and current glucose levels. The prediction visualization is configured to be adjustable based on user input and the visualization is configured to dynamically update based on user input. The disclosed interface allows the user to adjust the sequencing of these decisions and portion sizes of meal choices and immediately generate new visualizations representing the impact on predicted future glucose levels.

### CROSS-REFERENCE TO RELATED APPLICATIONS

This application claims priority to and the benefit of U.S. Provisional Application No. 63/589,477, filed Oct. 11, 2023, which is incorporated herein by reference in its entirety.

### BACKGROUND

Diabetics must regularly test their glucose levels. Knowledge of the glucose level is essential in helping a diabetic manage their diabetes. Diabetics monitor their blood sugar before meals, at bedtime, and at various other points throughout a day.

Conventionally, a diabetic determines their glucose level by pricking their fingertip with a needle and testing the blood drop with a glucose meter. Depending on the glucose level, the diabetic may adjust their behavior, e.g., medication intake, dietary choices, and exercise regimen. They may also provide this data to doctors and medical professionals.

Continuous glucose monitoring (“CGM”) systems have been developed that allow diabetics to monitor their glucose levels at regular intervals. CGM systems often rely on an under-the-skin, analyte sensor. An analyte is a substance whose chemical constituents are measured—in a CGM system, the analyte sensor measures the amount of glucose in body fluids. This obviates the need to draw blood with a needle and provides consistent, continuous readings. A coupled transmitter sends the readings to a wearable device, mobile device, or other external system. Glucose levels may be tracked every few seconds or minutes, 24 hours a day and 7 days a week.

A CGM system may analyze longer term trends. Exercise, diet, stress, and other life events may affect glucose levels. The data catalogued by a CGM system may lead to valuable insights. With this knowledge, a diabetic can determine their best diet and exercise regimen, adjust amounts of insulin and other medications, and develop an optimum plan to manage their diabetes. Various metrics have been developed to summarize a diabetic's glycemic status based on the readings, e.g., the ambulatory glucose profile report (“AGP”), glucose management indicator (“GMI”), time-in-range (“TIR”), time-above-180 (“TA180”), and others.

However, visualizations provided by legacy tools display only past glucose readings. For example, an AGP report displays the average glucose, a percentage of variability, time spent in ranges, and other statistics calculated across a selected date range. Legacy tools do not estimate future glucose levels or otherwise provide estimated future glucose levels in these visualizations.

### SUMMARY

Accordingly, a need exists to generate and display estimated future glucose levels in a CGM interface. This visualization may be referred to as the personal impact empathizer (“PIE”) or the “butterfly effect” visualization in this disclosure. The “butterfly effect” is a reference to the mathematics concept most often associated with mathematician Edward Norton Lorenz (and, of course, popularized by the fictional character Ian Malcom in Jurassic Park). The “butterfly effect” is the idea that a small change in one state of a deterministic nonlinear system can result in large differences in a later state. In the common, metaphorical example, a butterfly flapping its wings in one location may change the weather weeks later in a different location.

Because certain choices made by a diabetic at one time/state, e.g., diet and exercise, lead to changes in their glucose levels at a later time/state, the diabetic may benefit from understanding the “butterfly effect” of their choices. A PIE visualization may present predicted glucose-level data in continuity with past readings of glucose levels. This allows the user to view future data relative to their latest glucose profile. Future data may be presented as a single trace (i.e., a line), a range, or using another suitable approach. A PIE visualization makes the consequences of choices personally relatable in terms of their impact on the future glycemic profile. The subsystem responsible for the calculation of glucose may be referred to below as the glycemic profile emulation (“GPE”) subsystem.

Some legacy systems may allow users to tag events such as a meals and exercise. For example, a user may enter the type and amount of food eaten. Legacy systems may also allow a user to manually enter a physical activity or integrate with a wearable device to import past physical activities. But as discussed above, these legacy tools are deficient because these capabilities only examine past events. A user cannot enter future meals or future exercise events and analyze the impact on their future glycemic profile.

Moreover, legacy tools fail to offer the ability to change combinations and sequences of these events and view the impact of changes. Accordingly, a further need exists to allow users to assess the impact on future blood glucose levels of combinations of food choices and exercise and the order in which such events will occur. A technical benefit may be realized by allowing a user to assess the impact of sequences of such events by efficiently recording events, changing the sequences of the events, and quickly assessing the impact on blood glucose levels within the user interface.

Because users need to make life decisions with regularity, the amount of time and energy needed to track and log the information may be onerous, and any technical solution needs to be scalable and extendable. As such, a technical benefit may be realized by allowing the user to adjust the portion size of a future meal dynamically within the visualization and view the resulting predicted glucose level. A further optimization may be achieved by seamlessly integrating with partner systems and with wearable fitness devices. Such partner systems may include restaurants, meal delivery apps, diet programs, exercise programs, etc. These improvements may be scaled up using an appropriate application programming interfaces (“APIs”) for partner systems and by leveraging partner APIs.

According to one embodiment of the invention, a method for predicting the impact of personal choices on future analyte levels includes the steps of receiving analyte readings from an in vivo analyte sensor, displaying a hybrid sensor-meal visualization of the analyte readings on a mobile application running on a mobile device, receiving a food choice via a graphical user interface of the mobile application, calculating a personalized analyte prediction based on the food choice, user-specific parameters, and the analyte readings, and updating the visualization to display the personalized analyte prediction.

According to one aspect of some embodiments, the method may include retrieving one or more parameters associated with the food choice, wherein the one or more parameters comprise a glycemic index, a glycemic load, a rate of glucose appearance profile in an averaged person with standard food bioavailability, a macronutrient composition, and/or a fiber content. In one embodiment, the one or more parameters may be retrieved via a call to an application programming interface provided by a partner system. In one embodiment, the personalized analyte prediction displays as a single trace line and is presented graphically proximate to the visualization of the analyte readings.

### BRIEF DESCRIPTION OF THE DRAWINGS

The accompanying drawings, which are incorporated herein and form a part of the specification, illustrate embodiments of the present disclosure and, together with the description, further serve to explain the principles of the disclosure and to enable a person skilled in the arts to make and use the embodiments.

FIG. 1A is a system overview of a sensor applicator, reader device, monitoring system, network, and remote system.

FIG. 1B is a diagram illustrating an operating environment of an example analyte monitoring system for use with the techniques described herein.

FIG. 2A is a block diagram depicting an example embodiment of a reader device.

FIG. 2B is a block diagram illustrating an example data receiving device for communicating with the sensor according to exemplary embodiments of the disclosed subject matter.

FIGS. 2C and 2D are block diagrams depicting example embodiments of sensor control devices.

FIG. 2E is a block diagram illustrating an example analyte sensor according to exemplary embodiments of the disclosed subject matter.

FIG. 3A is a proximal perspective view depicting an example embodiment of a user preparing a tray for an assembly.

FIG. 3B is a side view depicting an example embodiment of a user preparing an applicator device for an assembly.

FIG. 3C is a proximal perspective view depicting an example embodiment of a user inserting an applicator device into a tray during an assembly.

FIG. 3D is a proximal perspective view depicting an example embodiment of a user removing an applicator device from a tray during an assembly.

FIG. 3E is a proximal perspective view depicting an example embodiment of a patient applying a sensor using an applicator device.

FIG. 3F is a proximal perspective view depicting an example embodiment of a patient with an applied sensor and a used applicator device.

FIG. 4A is a side view depicting an example embodiment of an applicator device coupled with a cap.

FIG. 4B is a side perspective view depicting an example embodiment of an applicator device and cap decoupled.

FIG. 4C is a perspective view depicting an example embodiment of a distal end of an applicator device and electronics housing.

FIG. 4D is a top perspective view of an exemplary applicator device in accordance with the disclosed subject matter.

FIG. 4E is a bottom perspective view of the applicator device of FIG. 4D.

FIG. 4F is an exploded view of the applicator device of FIG. 4D.

FIG. 4G is a side cutaway view of the applicator device of FIG. 4D.

FIG. 5 is a diagram illustrating example operational states of the sensor according to exemplary embodiments of the disclosed subject matter.

FIG. 6 is a diagram illustrating an example operational and data flow for over-the-air programming of a sensor according to the disclosed subject matter.

FIG. 7 is a diagram illustrating an example data flow for secure exchange of data between two devices according to the disclosed subject matter.

FIG. 8 is a block diagram of a CGM system that provides visualizations on the impact of personal choices on future glucose levels, according to some embodiments.

FIG. 9A is an example hybrid sensor-meal visualization of a CGM user interface including a visualization of past blood glucose levels, according to some embodiments.

FIG. 9B is an example hybrid sensor-meal visualization of a CGM user interface including a menu of options with a butterfly-effect selection, according to some embodiments.

FIG. 9C is an example hybrid sensor-meal visualization of a CGM user interface including a visualization of past blood glucose levels with an add-food button, according to some embodiments.

FIG. 10A is an example hybrid sensor-meal visualization of a CGM user interface including a visualization of single trace of estimated future glucose levels based on a food selection, according to some embodiments.

FIG. 10B is an example hybrid sensor-meal visualization of a CGM user interface including a visualization of ranges of estimated future glucose levels based on a food selection, according to some embodiments.

FIG. 11A is an example hybrid sensor-meal visualization of a CGM user interface including a visualization of estimated future glucose levels based on multiple food selections, according to some embodiments.

FIG. 11B is an example hybrid sensor-meal visualization of a CGM user interface including a visualization of estimated future glucose levels based on multiple food selections with a sequencing option, according to some embodiments.

FIG. 12A is an example hybrid sensor-meal visualization of a CGM user interface including a visualization of estimated future glucose levels based on multiple food selections with a portion controller, according to some embodiments.

FIG. 12B is an example hybrid sensor-meal visualization of a CGM user interface including a visualization of estimated future glucose levels based on multiple food selections with a movable portion controller, according to some embodiments.

FIG. 13A is an example hybrid sensor-meal visualization of a CGM user interface that displays heart rate and glucose levels, according to some embodiments.

FIG. 13B is an example hybrid sensor-meal visualization of a CGM user interface that displays step counts and glucose levels, according to some embodiments.

FIG. 14 is a flowchart illustrating a method of providing a personalized glucose prediction based on a food choice, according to some embodiments.

FIG. 15 is a flowchart illustrating a method of establishing a connection with a partner system to provide a personalized glucose prediction, according to some embodiments.

FIG. 16 is a flowchart illustrating a method of establishing a connection with a wearable to provide a personalized glucose prediction based on a physical activity, according to some embodiments.

FIG. 17 is a block diagram of models and profiles used to generate an emulated glycemic profile, according to some embodiments.

FIG. 18 is an example computer system useful for implementing various embodiments.

In the drawings, like reference numbers generally indicate identical or similar elements. Additionally, generally, the left-most digit(s) of a reference number identifies the drawing in which the reference number first appears.

### DETAILED DESCRIPTION

Provided herein are system, apparatus, device, method and/or computer program product embodiments, and/or combinations and sub-combinations thereof, for predicting the impact of personal choices on future glucose levels. The disclosed techniques assess the potential consequences of decisions involving diet and exercise on future glucose levels using trained models and display personally relatable, intuitive, and modifiable visualizations of predicted future glucose levels.

Several embodiments are disclosed herein for achieving an improved predictive visualization of future glucose levels through the use of an improved user interface that provides hybrid user interface components for viewing a combination of sensor data, predictive user information, and third-party data sources. In one embodiment, the hybrid sensor-meal visualization may include a PIE visualization. For example, in one embodiment, the hybrid sensor-meal visualization may be implemented as part of an independent application visually embedded into the CGM system application user interface (“UI”) with the ability to receive sensor data from a connected sensor device of the CGM system, user data related to food choice and physical activity from connected partner systems (e.g., via wellness apps, via partner app, via QR code from food menu, etc.). In this embodiment, the PIE includes user interface components for receiving user input for editing food choices (e.g., in terms of amount, order, and/or timing) and physical activity (e.g., in terms of type, intensity, order, and/or duration), and the GPE projects a near future glycemic profile starting from the latest user's glucose readings. The projection of near future glycemic profile may be presented in a graph similar to the current CGM trace containing historic glucose and current glucose.

In another embodiment, the PIE is an independent application that is visually separate from the CGM system application's user interface. In yet another embodiment, the PIE may be a sub-module integrated into the CGM system's application and visually embedded into the CGM system application's UI flow. In another embodiment, the PIE may provide one or more values to a connected wearable to provide direct feedback regarding the duration and intensity of the chosen physical activity. For example, the connected wearable may inform the user that a current period of exercise exceeds a length of time, heart rate, or other suitable indicator and/or creates a calculated near-future glucose estimate that exceeds a threshold. This allows the user to avoid a near-future glucose trajectory after the physical activity having a significantly higher or lower glucose than predicted at the start of the physical activity—i.e., a user may ensure that their actual physical activity follows a projection based on a chosen physical activity. Upper and lower ranges may provide a general indication on whether the resulting post-exercise glucose will be lower or higher than the predicted near future glucose prior to the start of the physical activity.

Before the present subject matter is described in detail, it is to be understood that this disclosure is not limited to the particular embodiments described, as such may, of course, vary. It is also to be understood that the terminology used herein is for the purpose of describing particular embodiments only, and is not intended to be limiting, since the scope of the present disclosure will be limited only by the appended claims.

As used herein and in the appended claims, the singular forms “a,” “an,” and “the” include plural referents unless the context clearly dictates otherwise.

The publications discussed herein are provided solely for their disclosure prior to the filing date of the present application. Nothing herein is to be construed as an admission that the present disclosure is not entitled to antedate such publication by virtue of prior disclosure. Further, the dates of publication provided may be different from the actual publication dates which may need to be independently confirmed.

Generally, embodiments of the present disclosure include systems, devices, and methods for the use of analyte sensor insertion applicators for use with in vivo analyte monitoring systems. An applicator can be provided to the user in a sterile package with an electronics housing of the sensor control device contained therein. According to some embodiments, a structure separate from the applicator, such as a container, can also be provided to the user as a sterile package with a sensor module and a sharp module contained therein. The user can couple the sensor module to the electronics housing, and can couple the sharp to the applicator with an assembly process that involves the insertion of the applicator into the container in a specified manner. In other embodiments, the applicator, sensor control device, sensor module, and sharp module can be provided in a single package. The applicator can be used to position the sensor control device on a human body with a sensor in contact with the wearer's bodily fluid. The embodiments provided herein are improvements to reduce the likelihood that a sensor is improperly inserted or damaged, or elicits an adverse physiological response. Other improvements and advantages are provided as well. The various configurations of these devices are described in detail by way of the embodiments which are only examples.

Furthermore, many embodiments include in vivo analyte sensors structurally configured so that at least a portion of the sensor is, or can be, positioned in the body of a user to obtain information about at least one analyte of the body. It should be noted, however, that the embodiments disclosed herein can be used with in vivo analyte monitoring systems that incorporate in vitro capability, as well as purely in vitro or ex vivo analyte monitoring systems, including systems that are entirely non-invasive.

Furthermore, for each and every embodiment of a method disclosed herein, systems and devices capable of performing each of those embodiments are covered within the scope of the present disclosure. For example, embodiments of sensor control devices are disclosed and these devices can have one or more sensors, analyte monitoring circuits (e.g., an analog circuit), memories (e.g., for storing instructions), power sources, communication circuits, transmitters, receivers, processors and/or controllers (e.g., for executing instructions) that can perform any and all method steps or facilitate the execution of any and all method steps. These sensor control device embodiments can be used and can be capable of use to implement those steps performed by a sensor control device from any and all of the methods described herein.

Furthermore, the systems and methods presented herein can be used for operations of a sensor used in an analyte monitoring system, such as but not limited to wellness, fitness, dietary, research, information or any purposes involving analyte sensing over time. As used herein, “analyte sensor” or “sensor” can refer to any device capable of receiving sensor information from a user, including for purpose of illustration but not limited to, body temperature sensors, blood pressure sensors, pulse or heart-rate sensors, glucose level sensors, analyte sensors, physical activity sensors, body movement sensors, or any other sensors for collecting physical or biological information. Analytes measured by the analyte sensors can include, by way of example and not limitation, glucose, ketones, lactate, oxygen, hemoglobin A1C, albumin, alcohol, alkaline phosphatase, alanine transaminase, aspartate aminotransferase, bilirubin, blood urea nitrogen, calcium, carbon dioxide, chloride, creatinine, hematocrit, lactate, magnesium, oxygen, pH, phosphorus, potassium, sodium, total protein, uric acid, etc.

As mentioned, a number of embodiments of systems, devices, and methods are described herein that provide for the improved assembly and use of dermal sensor insertion devices for use with in vivo analyte monitoring systems. In particular, several embodiments of the present disclosure are designed to improve the method of sensor insertion with respect to in vivo analyte monitoring systems and, in particular, to prevent the premature retraction of an insertion sharp during a sensor insertion process. Some embodiments, for example, include a dermal sensor insertion mechanism with an increased firing velocity and a delayed sharp retraction. In other embodiments, the sharp retraction mechanism can be motion-actuated such that the sharp is not retracted until the user pulls the applicator away from the skin. Consequently, these embodiments can reduce the likelihood of prematurely withdrawing an insertion sharp during a sensor insertion process; decrease the likelihood of improper sensor insertion; and decrease the likelihood of damaging a sensor during the sensor insertion process, to name a few advantages. Several embodiments of the present disclosure also provide for improved insertion sharp modules to account for the small scale of dermal sensors and the relatively shallow insertion path present in a subject's dermal layer. In addition, several embodiments of the present disclosure are designed to prevent undesirable axial and/or rotational movement of applicator components during sensor insertion. Accordingly, these embodiments can reduce the likelihood of instability of a positioned dermal sensor, irritation at the insertion site, damage to surrounding tissue, and breakage of capillary blood vessels resulting in fouling of the dermal fluid with blood, to name a few advantages. In addition, to mitigate inaccurate sensor readings which can be caused by trauma at the insertion site, several embodiments of the present disclosure can reduce the end-depth penetration of the needle relative to the sensor tip during insertion.

Before describing these aspects of the embodiments in detail, however, it is first desirable to describe examples of devices that can be present within, for example, an in vivo analyte monitoring system, as well as examples of their operation, all of which can be used with the embodiments described herein.

There are various types of in vivo analyte monitoring systems. CGM systems, for example, can transmit data from a sensor control device to a reader device continuously without prompting, e.g., automatically according to a schedule. “Flash Analyte Monitoring” systems (or “Flash Glucose Monitoring” systems or simply “Flash” systems), as another example, can transfer data from a sensor control device in response to a scan or request for data by a reader device, such as with a Near Field Communication (“NFC”) or Radio Frequency Identification (“RFID”) protocol. In vivo analyte monitoring systems can also operate without the need for finger stick calibration.

In vivo analyte monitoring systems can be differentiated from in vitro systems that contact a biological sample outside of the body (or ex vivo) and that typically include a meter device that has a port for receiving an analyte test strip carrying bodily fluid of the user, which can be analyzed to determine the user's blood sugar level.

In vivo monitoring systems can include a sensor that, while positioned in vivo, makes contact with the bodily fluid of the user and senses the analyte levels contained therein. The sensor can be part of the sensor control device that resides on the body of the user and contains the electronics and power supply that enable and control the analyte sensing. The sensor control device, and variations thereof, can also be referred to as a “sensor control unit,” an “on-body electronics” device or unit, an “on-body” device or unit, or a “sensor data communication” device or unit, to name a few.

In vivo monitoring systems can also include a device that receives sensed analyte data from the sensor control device and processes and/or displays that sensed analyte data, in any number of forms, to the user. This device, and variations thereof, can be referred to as a “handheld reader device,” “reader device” (or simply a “reader”), “handheld electronics” (or simply a “handheld”), a “portable data processing” device or unit, a “data receiver,” a “receiver” device or unit (or simply a “receiver”), or a “remote” device or unit, to name a few. Other devices such as personal computers have also been utilized with or incorporated into in vivo and in vitro monitoring systems.

### Exemplary In Vivo Analyte Monitoring System

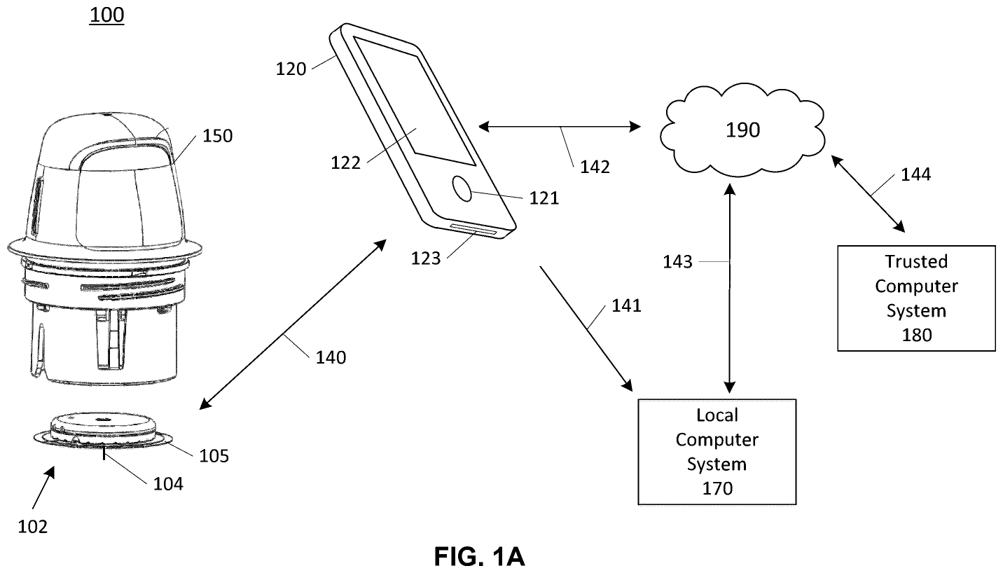

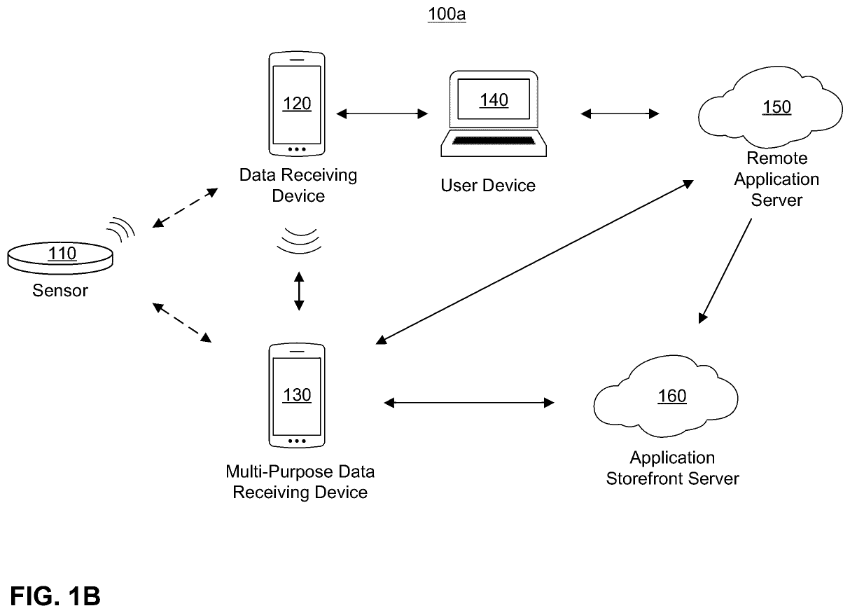

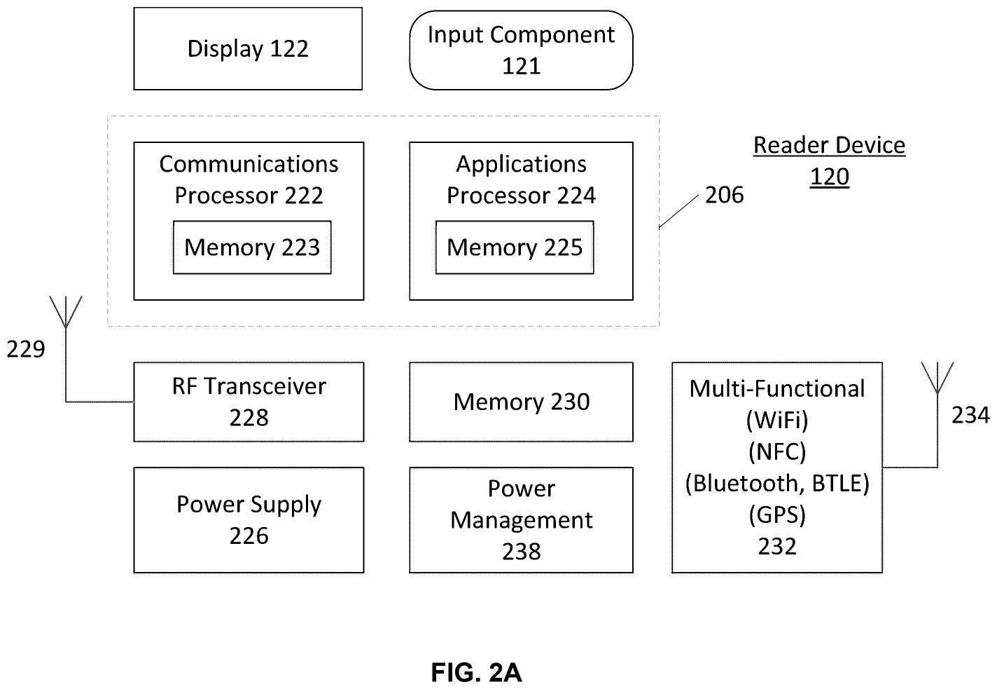

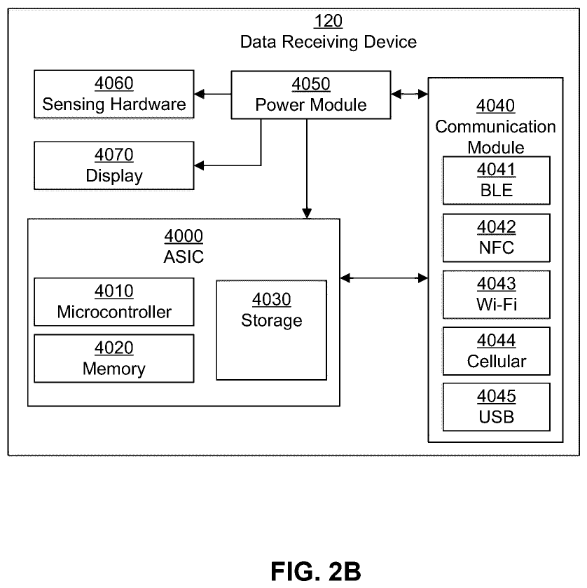

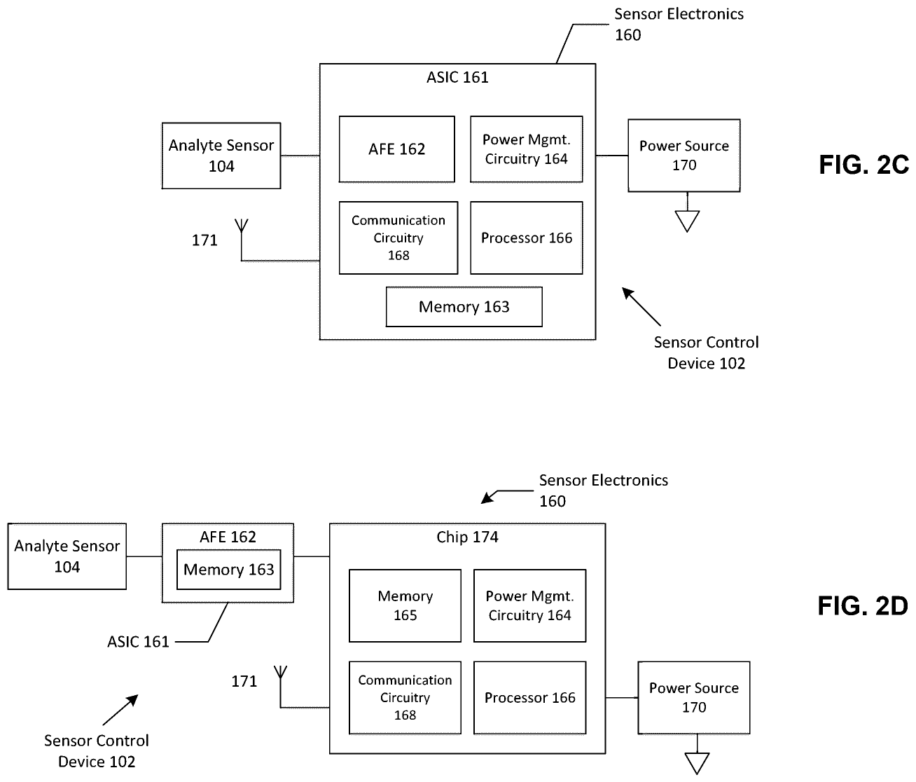

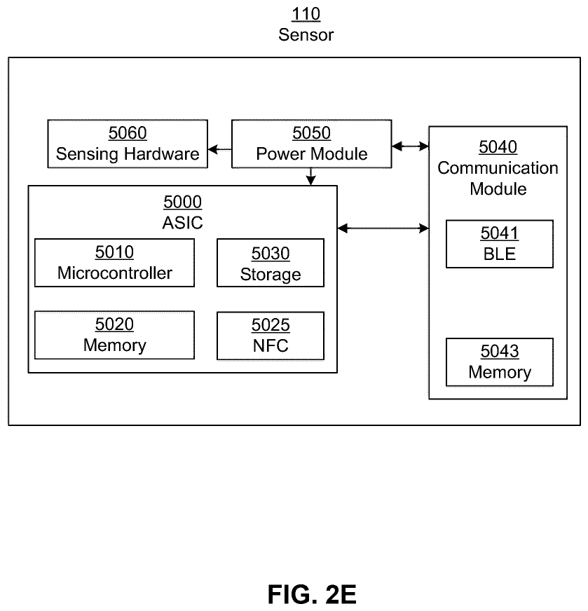

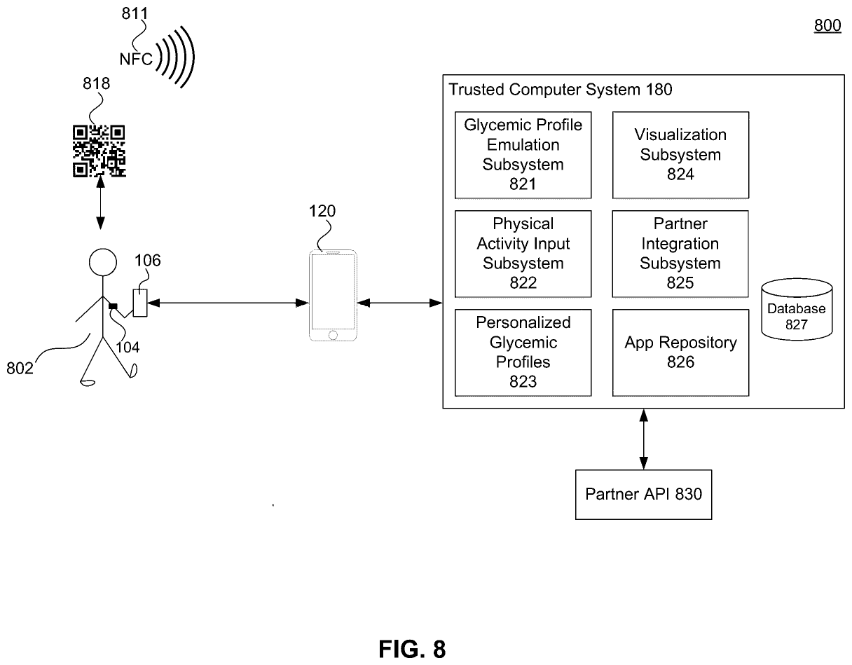

FIG. 1A is a conceptual diagram depicting an example embodiment of an analyte monitoring system 100 that includes applicator device 150, sensor control device 102, and data receiving device 120. Here, applicator device 150 can be used to deliver sensor control device 102 to a monitoring location on a user's skin where in vivo analyte sensor 104 is maintained in position for a period of time by adhesive patch 105. Sensor control device 102 is further described in FIGS. 2B and 2C, and can communicate with data receiving device 120 via a communication path 141 using a wired or wireless technique. Example wireless protocols include Bluetooth, Bluetooth Low Energy (“BLE” or “BTLE”), Bluetooth SMART, NFC, and others. Users can monitor applications installed in memory on data receiving device 120 using display 122 and input component 121 and the device battery can be recharged using power port 123. More detail about data receiving device 120 is set forth with respect to FIG. 2A below. Data receiving device 120 can communicate with local computer system 175 via a communication path 141 using a wired or wireless technique. Local computer system 175 can include one or more of a laptop, desktop, tablet, phablet, smartphone, set-top box, video game console, or other computing device and wireless communication can include any of a number of applicable wireless networking protocols including Bluetooth, BTLE, Wi-Fi or others. Local computer system 175 can communicate via communications path 143 with a network 190 similar to how data receiving device 120 can communicate via a communications path 142 with network 190, by wired or wireless technique as described previously. Network 190 can be any of a number of networks, such as private networks and public networks, local area or wide area networks, and so forth. A trusted computer system 180 can include a server and can provide authentication services and secured data storage and can communicate via communications path 144 with network 190 by wired or wireless technique. Trusted computer system 180 is described in further detail below with reference to FIG. 8.

FIG. 1B illustrates an operating environment of analyte monitoring system 100a capable of embodying the techniques described herein. Analyte monitoring system 100a may include a system of components designed to provide monitoring of parameters, such as analyte levels, of a human or animal body or can provide for other operations based on the configurations of the various components. As embodied herein, the system may include analyte sensor 110, or simply “sensor” worn by the user or attached to the body for which information is being collected. As embodied herein, analyte sensor 110 can be a sealed, disposable device with a predetermined active use lifetime (e.g., 1 day, 14 days, 30 days, etc.). Analyte sensor 110 may be applied to the skin of the user body and remain adhered over the duration of the sensor lifetime or can be designed to be selectively removed and remain functional when reapplied. Analyte monitoring system 100a can further include data receiving device 120 or multi-purpose data receiving device 130 configured as described herein to facilitate retrieval and delivery of data, including analyte data, from analyte sensor 110.

As embodied herein, analyte monitoring system 100a can include a software or firmware library or application provided, for example via remote application server 155 or application storefront server 160, to a third-party and incorporated into multi-purpose data receiving device 130 such as a mobile phone, tablet, personal computing device, or other similar computing device capable of communicating with analyte sensor 110 over a communication link. Multi-purpose hardware can further include embedded devices, including, but not limited to insulin pumps or insulin pens, having an embedded library configured to communicate with analyte sensor 110. Although the illustrated embodiments of analyte monitoring system 100a include only one of each of the illustrated devices, this disclosure contemplates analyte monitoring system 100a incorporating multiples of each component interacting throughout the system. For example and without limitation, as embodied herein, data receiving device 120 and/or multi-purpose data receiving device 130 can include multiples of each. As embodied herein, multi-purpose data receiving device 130 can communicate directly with analyte sensor 110 as described herein. Additionally or alternatively, multi-purpose data receiving device 130 can communicate with multi-purpose data receiving device 130 to provide analyte data, or visualization or analysis of the data, for secondary display to the user or other authorized parties.

### Exemplary Over-the-Air Updates

FIG. 1B further illustrates an example operating environment for providing over-the-air (“OTA”) updates for use with the techniques described herein. An operator of analyte monitoring system 100a can bundle updates for data receiving device 120 or analyte sensor 110 into updates for an application executing on multi-purpose data receiving device 130. Using available communication channels between data receiving device 120, multi-purpose data receiving device 130, and analyte sensor 110, multi-purpose data receiving device 130 can receive regular updates for data receiving device 120 or analyte sensor 110 and initiate installation of the updates on data receiving device 120 or analyte sensor 110. Multi-purpose data receiving device 130 acts as an installation or update platform for data receiving device 120 or analyte sensor 110 because the application that enables the multi-purpose data receiving device 130 to communicate with analyte sensor 110, data receiving device 120 and/or remote application server 155 can update software or firmware on data receiving device 120 or analyte sensor 110 without wide-area networking capabilities.

As embodied herein, remote application server 155 operated by the manufacturer of analyte sensor 110 and/or the operator of analyte monitoring system 100 can provide software and firmware updates to the devices of analyte monitoring system 100. In particular embodiments, remote application server 155 can provides the updated software and firmware to user device 140 or directly to a multi-purpose data receiving device. As embodied herein, remote application server 155 can also provide application software updates to application storefront server 160 using interfaces provided by the application storefront. Multi-purpose data receiving device 130 can contact the application storefront server 160 periodically to download and install the updates.

After multi-purpose data receiving device 130 downloads an application update including a firmware or software update for data receiving device 120 or analyte sensor 110, data receiving device 120 or analyte sensor 110 and multi-purpose data receiving device 130 establish a connection. Multi-purpose data receiving device 130 determines that a firmware or software update is available for data receiving device 120 or analyte sensor 110. Multi-purpose data receiving device 130 can prepare the software or firmware update for delivery to data receiving device 120 or analyte sensor 110. As an example, multi-purpose data receiving device 130 can compress or segment the data associated with the software or firmware update, can encrypt or decrypt the firmware or software update, or can perform an integrity check of the firmware or software update. Multi-purpose data receiving device 130 sends the data for the firmware or software update to data receiving device 120 or analyte sensor 110. Multi-purpose data receiving device 130 can also send a command to data receiving device 120 or analyte sensor 110 to initiate the update. Additionally or alternatively, multi-purpose data receiving device 130 can provide a notification to the user of multi-purpose data receiving device 130 and include instructions for facilitating the update, such as instructions to keep data receiving device 120 and multi-purpose data receiving device 130 connected to a power source and in close proximity until the update is complete.

Data receiving device 120 or analyte sensor 110 receives the data for the update and the command to initiate the update from multi-purpose data receiving device 130. Data receiving device 120 can then install the firmware or software update. To install the update, data receiving device 120 or analyte sensor 110 can place or restart itself in a so-called “safe” mode with limited operational capabilities. Once the update is completed, data receiving device 120 or analyte sensor 110 re-enters or resets into a standard operational mode. Data receiving device 120 or analyte sensor 110 can perform one or more self-tests to determine that the firmware or software update was installed successfully. Multi-purpose data receiving device 130 can receive the notification of the successful update. Multi-purpose data receiving device 130 can then report a confirmation of the successful update to remote application server 155.

In particular embodiments, storage memory 5030 of analyte sensor 110 in FIG. 2E includes OTP memory. The term OTP memory can refer to memory that includes access restrictions and security to facilitate writing to particular addresses or segments in the memory a predetermined number of times. Memory 5030 can be prearranged into multiple pre-allocated memory blocks or containers. The containers are pre-allocated into a fixed size. If storage memory 5030 is OTP memory, the containers can be considered to be in a non-programmable state. Additional containers which have not yet been written to can be placed into a programmable or writable state. Containerizing storage memory 5030 in this fashion can improve the transportability of code and data to be written to storage memory 5030. Updating the software of a device (e.g., the sensor device described herein) stored in an OTP memory can be performed by superseding only the code in a particular previously-written container or containers with updated code written to a new container or containers, rather than replacing the entire code in the memory. In a second embodiment, the memory is not prearranged. Instead, the space allocated for data is dynamically allocated or determined as needed. Incremental updates can be issued, as containers of varying sizes can be defined where updates are anticipated.

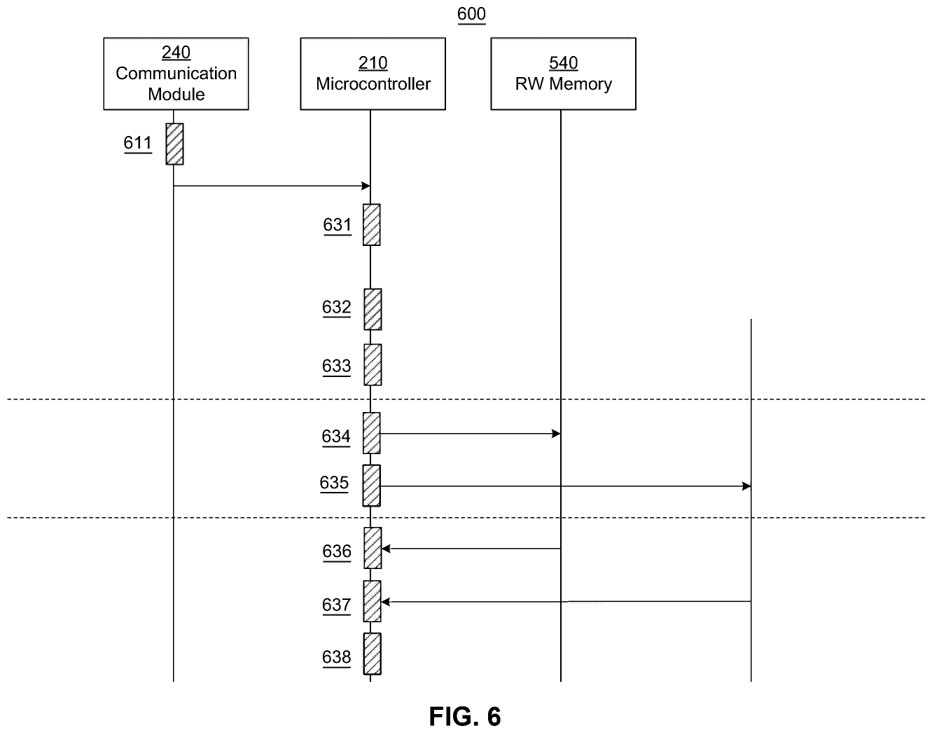

FIG. 6 is a diagram illustrating an example operational and data flow for over-the-air (“OTA”) programming of storage memory 5030 in analyte monitoring system 100 as well as use of the memory after the OTA programming in execution of processes by analyte sensor 110 according to the disclosed subject matter. In the example OTA programming 600 illustrated in FIG. 6, a request is sent from an external device (e.g., multi-purpose data receiving device 130) to initiate OTA programming (or re-programming). At 611, communication module 240 of analyte sensor 110 receives an OTA programming command. Communication module 240 sends the OTA programming command to the microcontroller 210 of analyte sensor 110.

At 631, after receiving the OTA programming command, microcontroller 210 validates the OTA programming command. Microcontroller 210 can determine, for example, whether the OTA programming command is signed with an appropriate digital signature token. Upon determining that the OTA programming command is valid, microcontroller 210 can set the sensor device into an OTA programming mode. At 632, microcontroller 210 can validate the OTA programming data. At 633, microcontroller 210 can reset analyte sensor 110 to re-initialize analyte sensor 110 in a programming state. Once analyte sensor 110 has transitioned into the OTA programming state, microcontroller 210 can begin to write data to the rewriteable memory (e.g., memory 5020) of the sensor device at 634 and write data to OTP memory 550 of the sensor device at 635 (e.g., storage memory 5030). The data written by the microcontroller 210 can be based on the validated OTA programming data. Microcontroller 210 can write data to cause one or more programming blocks or regions of OTP memory 550 to be marked invalid or inaccessible. The data written to the free or unused portion of OTP memory 550 can be used to replace invalidated or inaccessible programming blocks of OTP memory 550. After microcontroller 210 writes the data to the respective memories at 634 and 635, microcontroller 210 can perform one or more software integrity checks to ensure that errors were not introduced into the programming blocks during the writing process. Once microcontroller 210 is able to determine that the data has been written without errors, microcontroller 210 can resume standard operations of the sensor device.

In execution mode, at 636, microcontroller 210 can retrieve a programming manifest or profile from the rewriteable memory. The programming manifest or profile can include a listing of the valid software programming blocks and can include a guide to program execution for analyte sensor 110. By following the programming manifest or profile, microcontroller 210 can determine which memory blocks of OTP memory 550 are appropriate to execute and avoid execution of out-of-date or invalidated programming blocks or reference to out-of-date data. At 637, microcontroller 210 can selectively retrieve memory blocks from OTP memory 550. At 638, microcontroller 210 can use the retrieved memory blocks, by executing programming code stored or using variable stored in the memory.

### Exemplary Reader Device

FIG. 2A is a block diagram depicting an example embodiment of a reader device configured as a smartphone. Here, data receiving device 120 can include a display 122, input component 121, and processing core 206 including communications processor 222 coupled with memory 223 and applications processor 224 coupled with memory 225. Also included can be separate memory 230, RF transceiver 228 with antenna 229, and power supply 226 with power management module 238. Further included can be a multi-functional transceiver 232 which can communicate over Wi-Fi, NFC, Bluetooth, BTLE, and GPS with antenna 234. As understood by one of skill in the art, these components are electrically and communicatively coupled in a manner to make a functional device.

### Exemplary Data Receiving Device Architecture

For purpose of illustration and not limitation, reference is made to the exemplary embodiment of data receiving device 120 for use with the disclosed subject matter as shown in FIG. 2B. Data receiving device 120 and the related multi-purpose data receiving device 130 include components germane to the discussion of analyte sensor 110 and its operations and additional components can be included. In particular embodiments, data receiving device 120 and multi-purpose data receiving device 130 can be or include components provided by a third party and are not necessarily restricted to include devices made by the same manufacturer as analyte sensor 110.

As illustrated in FIG. 2B, data receiving device 120 includes ASIC 4000 having microcontroller 4010, memory 4020, and storage 4030 and communicatively coupled with a communication module 4040. Power for the components of data receiving device 120 can be delivered by power module 4050, which as embodied herein can include a rechargeable battery. Data receiving device 120 can further include display 4070 for facilitating review of analyte data received from analyte sensor 110 or other device (e.g., user device 140 or remote application server 155). Data receiving device 120 can include separate user interface components (e.g., physical keys, light sensors, microphones, etc.).

Communication module 4040 can include BLE module 4041 and NFC module 4042. Data receiving device 120 can be configured to wirelessly couple with analyte sensor 110 and transmit commands to and receive data from analyte sensor 110. As embodied herein, data receiving device 120 can be configured to operate, with respect to analyte sensor 110 as described herein, as an NFC scanner and a BLE end point via specific modules (e.g., BLE module 4042 or NFC module 4043) of communication module 4040. For example, data receiving device 120 can issue commands (e.g., activation commands for a data broadcast mode of the sensor; pairing commands to identify data receiving device 120) to analyte sensor 110 using a first module of the communication module 4040 and receive data from and transmit data to analyte sensor 110 using a second module of the communication module 4040. Data receiving device 120 can be configured for communication with user device 140 via a Universal Serial Bus (“USB”) module 4045 of the communication module 4040.

As another example, communication module 4040 can include, for example, cellular radio module 4044. The cellular radio module 4044 can include one or more radio transceivers for communicating using broadband cellular networks, including, but not limited to third generation (“3G”), fourth generation (“4G”), and fifth generation (“5G”) networks. Additionally, communication module 4040 of data receiving device 120 can include Wi-Fi radio module 4043 for communication using a wireless local area network according to one or more of the IEEE 802.11 standards (e.g., 802.11a, 802.11b, 802.11g, 802.11n (aka Wi-Fi 4), 802.11ac (aka Wi-Fi 5), 802.11ax (aka Wi-Fi 6)). Using cellular radio module 4044 or Wi-Fi radio module 4043, data receiving device 120 can communicate with remote application server 155 to receive analyte data or provide updates or input received from a user (e.g., through one or more user interfaces). Although not illustrated, communication module 5040 of analyte sensor 110 can similarly include a cellular radio module or Wi-Fi radio module.

As embodied herein, on-board storage 4030 of data receiving device 120 can store analyte data received from analyte sensor 110. Further, data receiving device 120, multi-purpose data receiving device 130, or user device 140 can be configured to communicate with remote application server 155 via a wide area network. As embodied herein, analyte sensor 110 can provide data to data receiving device 120 or multi-purpose data receiving device 130. Data receiving device 120 can transmit the data to user device 140. User device 140 (or multi-purpose data receiving device 130) can in turn transmit that data to remote application server 155 for processing and analysis.

As embodied herein, data receiving device 120 can further include sensing hardware 4060 similar to, or expanded from, sensing hardware 5060 of analyte sensor 110. In particular embodiments, data receiving device 120 can be configured to operate in coordination with analyte sensor 110 and based on analyte data received from the analyte sensor 110. As an example, where analyte sensor 110 is a glucose sensor, data receiving device 120 can be or include an insulin pump or insulin injection pen. In coordination, multi-purpose data receiving device 130 can adjust an insulin dosage for a user based on glucose values received from the analyte sensor.

### Exemplary Sensor Control Devices

FIGS. 2C and 2D are block diagrams depicting example embodiments of sensor control device 102 having in vivo analyte sensor 104 and sensor electronics 169 (including analyte monitoring circuitry) that can have the majority of the processing capability for rendering end-result data suitable for display to the user. In FIG. 2C, a single semiconductor chip 161 is depicted that can be a custom application specific integrated circuit (“ASIC”). Shown within ASIC 161 are certain high-level functional units, including an analog front end (“AFE”) 162, power management circuitry 164, processor 166, and communication circuitry 168 (which can be implemented as a transmitter, receiver, transceiver, passive circuit, or otherwise according to the communication protocol). In this embodiment, both AFE 162 and processor 166 are used as analyte monitoring circuitry, but in other embodiments either circuit can perform the analyte monitoring function. Processor 166 can include one or more processors, microprocessors, controllers, and/or microcontrollers, each of which can be a discrete chip or distributed amongst (and a portion of) a number of different chips.

Memory 163 is also included within ASIC 161 and can be shared by the various functional units present within ASIC 161, or can be distributed amongst two or more of them. Memory 163 can also be a separate chip. Memory 163 can be volatile and/or non-volatile memory. In this embodiment, ASIC 161 is coupled with power source 170, which can be a coin cell battery, or the like. AFE 162 interfaces with in vivo analyte sensor 104 and receives measurement data therefrom and outputs the data to processor 166 in digital form, which in turn processes the data to arrive at the end-result glucose discrete and trend values, etc. This data can then be provided to communication circuitry 168 for sending, by way of antenna 171, to data receiving device 120 (not shown), for example, where minimal further processing is needed by the resident software application to display the data.

FIG. 2D is similar to FIG. 2C but instead includes two discrete semiconductor chips 162 and 174, which can be packaged together or separately. Here, AFE 162 is resident on ASIC 161. Processor 166 is integrated with power management circuitry 164 and communication circuitry 168 on chip 174. AFE 162 includes memory 163 and chip 174 includes memory 165, which can be isolated or distributed within. In one example embodiment, AFE 162 is combined with power management circuitry 164 and processor 166 on one chip, while communication circuitry 168 is on a separate chip. In another example embodiment, both AFE 162 and communication circuitry 168 are on one chip, and processor 166 and power management circuitry 164 are on another chip. It should be noted that other chip combinations are possible, including three or more chips, each bearing responsibility for the separate functions described, or sharing one or more functions for fail-safe redundancy.

For purpose of illustration and not limitation, reference is made to the exemplary embodiment of analyte sensor 110 for use with the disclosed subject matter as shown in FIG. 2E. FIG. 2E illustrates a block diagram of an example analyte sensor 110 according to exemplary embodiments compatible with the security architecture and communication schemes described herein.

As embodied herein, analyte sensor 110 can include ASIC 5000 communicatively coupled with a communication module 5040. ASIC 5000 can include a microcontroller core 5010, on-board memory 5020, and storage memory 5030. Storage memory 5030 can store data used in an authentication and encryption security architecture. Storage memory 5030 can store programming instructions for analyte sensor 110. As embodied herein, certain communication chipsets can be embedded in the ASIC 5000 (e.g., an NFC transceiver 5025). ASIC 5000 can receive power from a power module 5050, such as an on-board battery or from an NFC pulse. Storage memory 5030 of the ASIC 5000 can be programmed to include information such as an identifier for analyte sensor 110 for identification and tracking purposes. Storage memory 5030 can also be programmed with configuration or calibration parameters for use by analyte sensor 110 and its various components. Storage memory 5030 can include rewritable or one-time programming (“OTP”) memory. The storage memory 5030 can be updated using techniques described herein to extend the usefulness of analyte sensor 110.

As embodied herein, communication module 5040 of sensor 110 can be or include one or more modules to support analyte sensor 110 communicating with other devices of the analyte monitoring system 100. As an example only and not by way of limitation, example communication modules 5040 can include BLE module 5041 As used throughout this disclosure, BLE refers to a short-range communication protocol optimized to make pairing of Bluetooth devices simple for end users. Communication module 5040 can transmit and receive data and commands via interaction with similarly-capable communication modules of data receiving device 120 or user device 140. Communication module 5040 can include additional or alternative chipsets for use with similar short-range communication schemes, such as a personal area network according to IEEE 802.15 protocols, IEEE 802.11 protocols, infrared communications according to the Infrared Data Association standards (IrDA), etc.

To perform its functionalities, sensor 110 can further include suitable sensing hardware 5060 appropriate to its function. As embodied herein, sensing hardware 5060 can include an analyte sensor transcutaneously or subcutaneously positioned in contact with a bodily fluid of a subject. The analyte sensor can generate sensor data containing values corresponding to levels of one or more analytes within the bodily fluid.

### Exemplary Assembly Processes for Sensor Control Devices

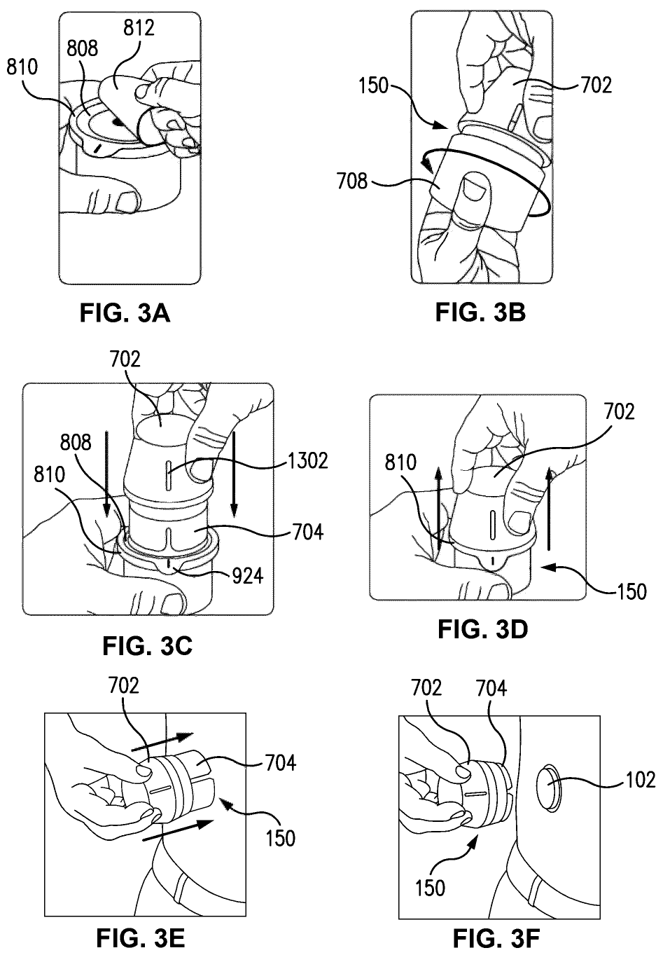

The components of sensor control device 102 can be acquired by a user in multiple packages requiring final assembly by the user before delivery to an appropriate user location. FIGS. 3A-3D depict an example embodiment of an assembly process for sensor control device 102 by a user, including preparation of separate components before coupling the components in order to ready the sensor for delivery. FIGS. 3E-3F depict an example embodiment of delivery of sensor control device 102 to an appropriate user location by selecting the appropriate delivery location and applying device 102 to the location.

FIG. 3A is a proximal perspective view depicting an example embodiment of user preparing tray 810, configured here as a tray (although other packages/containers can be used), for an assembly process. The user can accomplish this preparation by removing lid 812 from tray 810 to expose platform 808, for instance by peeling a non-adhered portion of lid 812 away from tray 810 such that adhered portions of lid 812 are removed. Removal of lid 812 can be appropriate in various embodiments so long as platform 808 is adequately exposed within tray 810. Lid 812 can then be placed aside.

FIG. 3B is a side view depicting an example embodiment of a user preparing an applicator device 150 for assembly. Applicator device 150 can be provided in a sterile package scaled by a cap 708. Preparation of applicator device 150 can include uncoupling housing 702 from cap 708 to expose sheath 704 (FIG. 3C). This can be accomplished by unscrewing (or otherwise uncoupling) cap 708 from housing 702. Cap 708 can then be placed aside.

FIG. 3C is a proximal perspective view depicting an example embodiment of a user inserting applicator device 150 into tray 810 during an assembly. Initially, the user can insert sheath 704 into platform 808 inside tray 810 after aligning a housing orienting feature (or slot or recess) and tray orienting feature 924 (an abutment or detent). Inserting sheath 704 into platform 808 temporarily unlocks sheath 704 relative to housing 702 and also temporarily unlocks platform 808 relative to tray 810. At this stage, removal of applicator device 150 from tray 810 will result in the same state prior to initial insertion of applicator device 150 into tray 810 (i.e., the process can be reversed or aborted at this point and then repeated without consequence).

Sheath 704 can maintain position within platform 808 with respect to housing 702 while housing 702 is distally advanced, coupling with platform 808 to distally advance platform 808 with respect to tray 810. This step unlocks and collapses platform 808 within tray 810. Sheath 704 can contact and disengage locking features (not shown) within tray 810 that unlock sheath 704 with respect to housing 702 and prevent sheath 704 from moving (relatively) while housing 702 continues to distally advance platform 808. At the end of advancement of housing 702 and platform 808, sheath 704 is permanently unlocked relative to housing 702. A sharp and sensor (not shown) within tray 810 can be coupled with an electronics housing (not shown) within housing 702 at the end of the distal advancement of housing 702. Operation and interaction of the applicator device 150 and tray 810 are further described below.

FIG. 3D is a proximal perspective view depicting an example embodiment of a user removing an applicator device 150 from tray 810 during an assembly. A user can remove applicator device 150 from tray 810 by proximally advancing housing 702 with respect to tray 810 or other motions having the same end effect of uncoupling applicator device 150 and tray 810. The applicator device 150 is removed with sensor control device 102 (not shown) fully assembled (sharp, sensor, electronics) therein and positioned for delivery.

FIG. 3E is a proximal perspective view depicting an example embodiment of a patient applying sensor control device 102 using applicator device 150 to a target area of skin, for instance, on an abdomen or any other appropriate location (e.g., on a limb). Advancing housing 702 distally collapses sheath 704 within housing 702 and applies the sensor to the target location such that an adhesive layer on the bottom side of sensor control device 102 adheres to the skin. The sharp is automatically retracted when housing 702 is fully advanced, while the sensor (not shown) is left in position to measure analyte levels.

FIG. 3F is a proximal perspective view depicting an example embodiment of a patient with sensor control device 102 in an example applied position. The user can then remove applicator device 150 from the application site.

Analyte monitoring system 100, described with respect to FIGS. 3A-3F and elsewhere herein, can provide a reduced or eliminated chance of accidental breakage, permanent deformation, or incorrect assembly of applicator components compared to prior art systems. Since housing 702 directly engages platform 808 while sheath 704 unlocks, rather than indirect engagement via sheath 704, relative angularity between sheath 704 and housing 702 will not result in breakage or permanent deformation of the arms or other components. The potential for relatively high forces (such as in conventional devices) during assembly will be reduced, which in turn reduces the chance of unsuccessful user assembly.

### Exemplary Sensor Applicator Devices

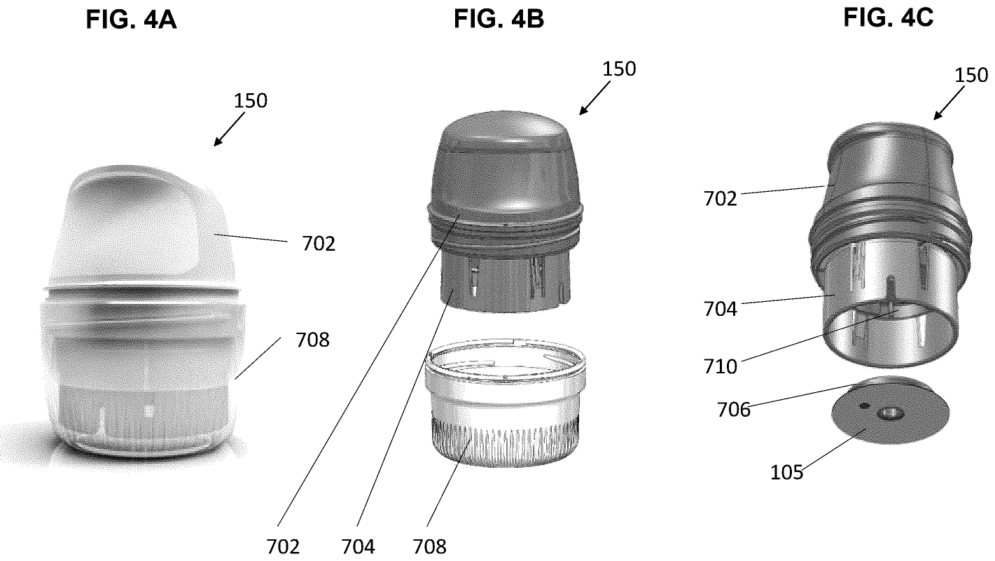

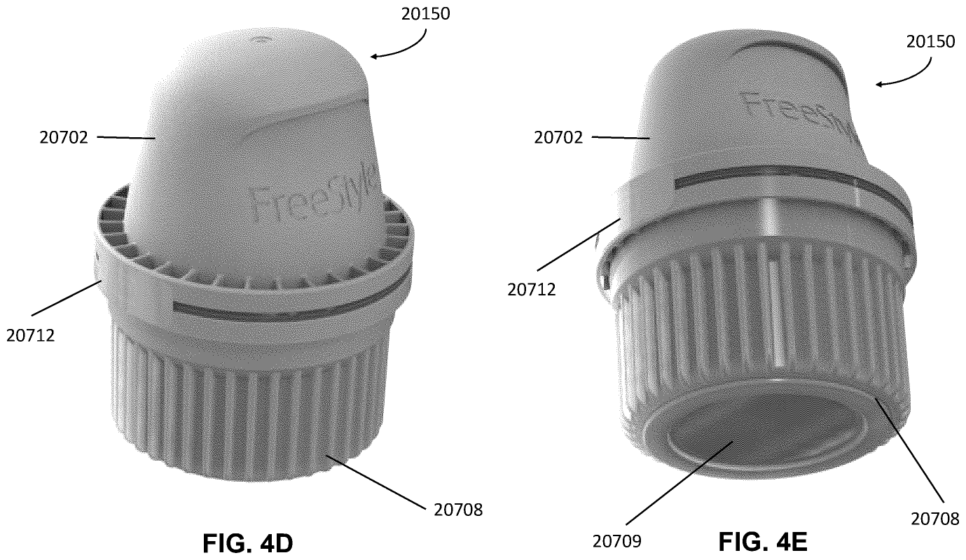

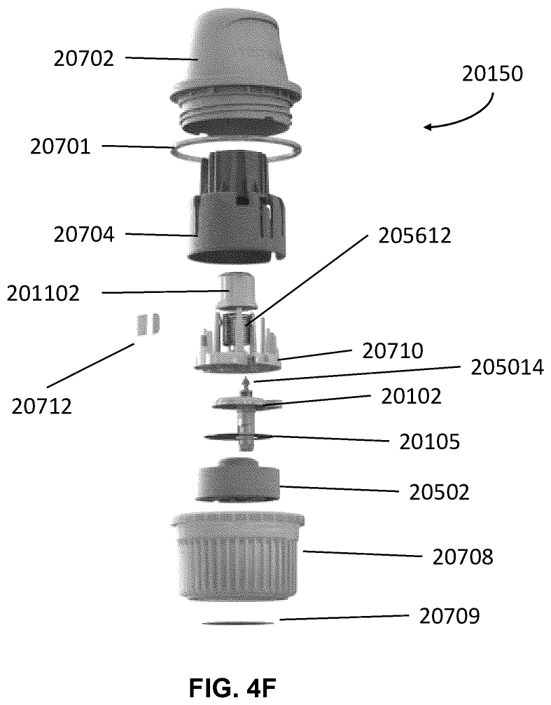

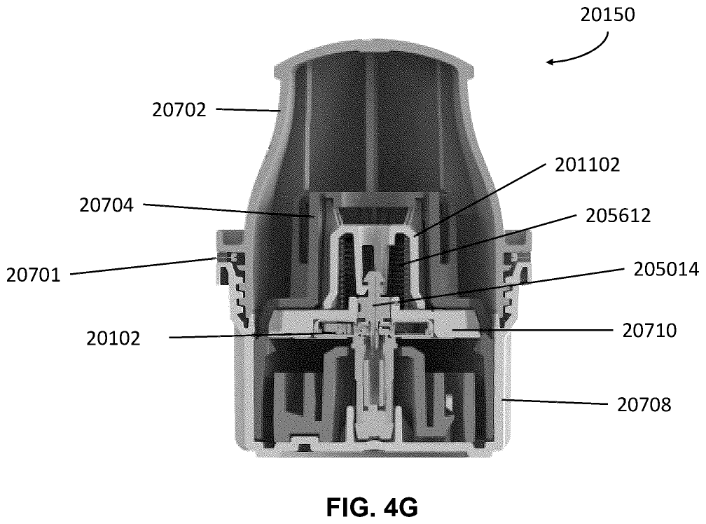

FIG. 4A is a side view depicting an example embodiment of an applicator device 150 coupled with screw cap 708. This is an example of how applicator device 150 is shipped to and received by a user, prior to assembly by the user with a sensor. FIG. 4B is a side perspective view depicting applicator device 150 and cap 708 after being decoupled. FIG. 4C is a perspective view depicting an example embodiment of a distal end of applicator device 150 with electronics housing 706 and adhesive patch 105 removed from the position they would have retained within sensor carrier 710 of sheath 704, when cap 708 is in place.

Referring to FIG. 4D-G for purpose of illustration and not limitation, applicator device 20150 can be provided to a user as a single integrated assembly. FIGS. 4D and 4E provide perspective top and bottom views, respectively, of the applicator device 20150, FIG. 4F provides an exploded view of applicator device 20150 and FIG. 4G provides a side cut-away view. The perspective views illustrate how applicator device 20150 is shipped to and received by a user. The exploded and cut-away views illustrate the components of applicator device 20150. The applicator device 20150 can include a housing 20702, gasket 20701, sheath 20704, sharp carrier 201102, spring 205612, sensor carrier 20710 (also referred to as a “puck carrier”), sharp hub 205014, sensor control device (also referred to as a “puck”) 20102, adhesive patch 20105, desiccant 20502, cap 20708, serial label 20709, and tamper evidence feature 20712. As received by a user, only housing 20702, cap 20708, tamper evidence feature 20712, and label 20709 are visible. The tamper evidence feature 20712 can be, for example, a sticker coupled to each of housing 20702 and cap 20708, and tamper evidence feature 20712 can be damaged, for example, irreparably, by uncoupling housing 20702 and cap 20708, thereby indicating to a user that housing 20702 and cap 20708 have been previously uncoupled. These features are described in greater detail below.

### Exemplary Sensor States and Activation

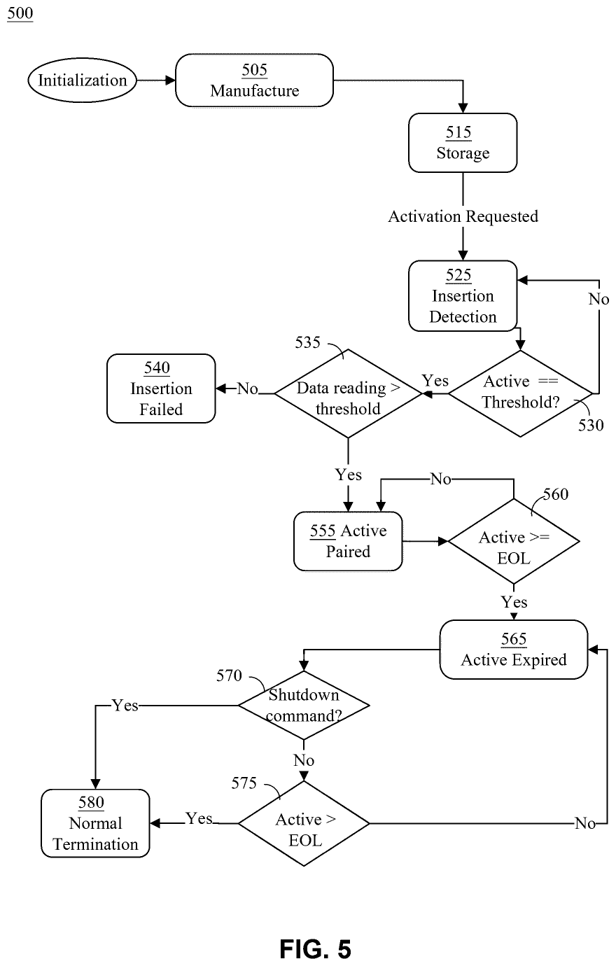

For purpose of illustration and not limitation, reference is made to the exemplary embodiment of a high-level depiction of a state machine representation 500 of the actions that can be taken by analyte sensor 110 as shown in FIG. 5. After initialization, the sensor enters state 505, which relates to the manufacture of analyte sensor 110. In the manufacture state 505, analyte sensor 110 can be configured for operation, for example, the storage memory 5030 can be written. At various times while in state 505, analyte sensor 110 checks for a received command to go to storage state 515. Upon entry to storage state 515, the sensor performs a software integrity check. While in storage state 515, the sensor can also receive an activation request command before advancing to insertion detection state 525.

Upon entry to state 525, analyte sensor 110 can store information relating to devices authenticated to communicate with the sensor as set during activation or initialize algorithms related to conducting and interpreting measurements from sensing hardware 5060. Analyte sensor 110 can also initialize a lifecycle timer, responsible for maintaining an active count of the time of operation of analyte sensor 110 and begin communication with authenticated devices to transmit recorded data. While in insertion detection state 525, the sensor can enter state 530, where analyte sensor 110 checks whether the time of operation is equal to a predetermined threshold. This time of operation threshold can correspond to a timeout function for determining whether an insertion has been successful. If the time of operation has reached the threshold, analyte sensor 110 advances to state 535, in which analyte sensor 110 checks whether the average data reading is greater than a threshold amount corresponding to an expected data reading volume for triggering detection of a successful insertion. If the data reading volume is lower than the threshold while in state 535, the sensor advances to state 540, corresponding to a failed insertion. If the data reading volume satisfies the threshold, the sensor advances to active paired state 555.

Active paired state 555 of analyte sensor 110 reflects the state while analyte sensor 110 is operating as normal by recording measurements, processing the measurements, and reporting them as appropriate. While in active paired state 555, analyte sensor 110 sends measurement results or attempts to establish a connection with data receiving device 120. Analyte sensor 110 also increments the time of operation. Once analyte sensor 110 reaches a predetermined threshold time of operation (e.g., once the time of operation reaches a predetermined threshold), analyte sensor 110 transitions to active expired state 565. Active expired state 565 of analyte sensor 110 reflects the state while analyte sensor 110 has operated for its maximum predetermined amount of time.

While in active expired state 565, analyte sensor 110 can generally perform operations relating to winding down operation and ensuring that the collected measurements have been securely transmitted to receiving devices as needed. For example, while in active expired state 565, analyte sensor 110 can transmit collected data and, if no connection is available, can increase efforts to discover authenticated devices nearby and establish and connection therewith. While in active expired state 565, analyte sensor 110 can receive a shutdown command at state 570. If no shutdown command is received, analyte sensor 110 can also, at state 575, check if the time of operation has exceeded a final operation threshold. The final operation threshold can be based on the battery life of analyte sensor 110. The normal termination state 580 corresponds to the final operations of analyte sensor 110 and ultimately shutting down analyte sensor 110.

Before a sensor is activated, ASIC 5000 resides in a low power storage mode state. The activation process can begin, for example, when an incoming RF field (e.g., NFC field) drives the voltage of the power supply to the ASIC 5000 above a reset threshold, which causes analyte sensor 110 to enter a wake-up state. While in the wake-up state, ASIC 5000 enters an activation sequence state. ASIC 5000 then wakes communication module 5040. Communication module 5040 is initialized, triggering a power on self-test. The power on self-test can include ASIC 5000 communicating with communication module 5040 using a prescribed sequence of reading and writing data to verify the memory and one-time programmable memory are not corrupted.

When ASIC 5000 enters the measurement mode for the first time, an insertion detection sequence is performed to verify that analyte sensor 110 has been properly installed onto the patient's body before a proper measurement can take place. First, analyte sensor 110 interprets a command to activate the measurement configuration process, causing the ASIC 5000 to enter measurement command mode. Analyte sensor 110 then temporarily enters the measurement lifecycle state to run a number of consecutive measurements to test whether the insertion has been successful. Communication module 5040 or ASIC 5000 evaluates the measurement results to determine insertion success. When insertion is deemed successful, analyte sensor 110 enters a measurement state, in which analyte sensor 110 begins taking regular measurements using sensing hardware 5060. If analyte sensor 110 determines that the insertion was not successful, analyte sensor 110 is triggered into an insertion failure mode, in which ASIC 5000 is commanded back to storage mode while communication module 5040 disables itself.

### Exemplary Security and Other Architecture Features

As embodied herein a first layer of security for communications between analyte sensor 110 and other devices can be established based on security protocols specified by and integrated in the communication protocols used for the communication. Another layer of security can be based on communication protocols that necessitate close proximity of communicating devices. Furthermore certain packets and/or certain data included within packets can be encrypted while other packets and/or data within packets is otherwise encrypted or not encrypted. Additionally or alternatively, application layer encryption can be used with one or more block ciphers or stream ciphers to establish mutual authentication and communication encryption with other devices in the analyte monitoring system 100.

ASIC 5000 of analyte sensor 110 in FIG. 2E can be configured to dynamically generate authentication and encryption keys using data retained within storage memory 5030. Storage memory 5030 can also be pre-programmed with a set of valid authentication and encryption keys to use with particular classes of devices. ASIC 5000 can be further configured to perform authentication procedures with other devices using received data and apply the generated key to sensitive data prior to transmitting the sensitive data. The generated key can be unique to analyte sensor 110, unique to a pair of devices, unique to a communication session between analyte sensor 110 and other device, unique to a message sent during a communication session, or unique to a block of data contained within a message.

Both analyte sensor 110 and data receiving device 120 can ensure the authorization of the other party in a communication session to, for example, issue a command or receive data. In particular embodiments, identity authentication can be performed through two features. First, the party asserting its identity provides a validated certificate signed by the manufacturer of the device or the operator of analyte monitoring system 100. Second, authentication can be enforced through the use of public keys and private keys, and shared secrets derived therefrom, established by the devices of analyte monitoring system 100 or established by the operator of the analyte monitoring system 100. To confirm the identity of the other party, the party can provide proof that the party has control of its private key.

The manufacturer of analyte sensor 110, data receiving device 120, or provider of the application for multi-purpose data receiving device 130 can provide information and programming necessary for the devices to securely communicate through secured programming and updates. For example, the manufacturer can provide information that can be used to generate encryption keys for each device, including secured root keys for analyte sensor 110 and optionally for data receiving device 120 that can be used in combination with device-specific information and operational data (e.g., entropy-based random values) to generate encryption values unique to the device, session, or data transmission as need.

Analyte data associated with a user is sensitive data at least in part because this information can be used for a variety of purposes, including for health monitoring and medication dosing decisions. In addition to user data, analyte monitoring system 100 can enforce security hardening against efforts by outside parties to reverse-engineering. Communication connections can be encrypted using a device-unique or session-unique encryption key. Encrypted communications or unencrypted communications between any two devices can be verified with transmission integrity checks built into the communications. Analyte sensor 110 operations can be protected from tampering by restricting access to read and write functions to the memory 5020 via a communication interface. The sensor can be configured to grant access only to known or “trusted” devices, provided in a “whitelist” or only to devices that can provide a predetermined code associated with the manufacturer or an otherwise authenticated user. A whitelist can represent an exclusive range, meaning that no connection identifiers besides those included in the whitelist will be used, or a preferred range, in which the whitelist is searched first, but other devices can still be used. Analyte sensor 110 can further deny and shut down connection requests if the requestor cannot complete a login procedure over a communication interface within a predetermined period of time (e.g., within four seconds). These characteristics safeguard against specific denial of service attacks, and in particular against denial of service attacks on a BLE interface.

As embodied herein, analyte monitoring system 100 can employ periodic key rotation to further reduce the likelihood of key compromise and exploitation. A key rotation strategy employed by analyte monitoring system 100 can be designed to support backward compatibility of field-deployed or distributed devices. As an example, analyte monitoring system 100 can employ keys for downstream devices (e.g., devices that are in the field or cannot be feasibly provided updates) that are designed to be compatible with multiple generations of keys used by upstream devices.

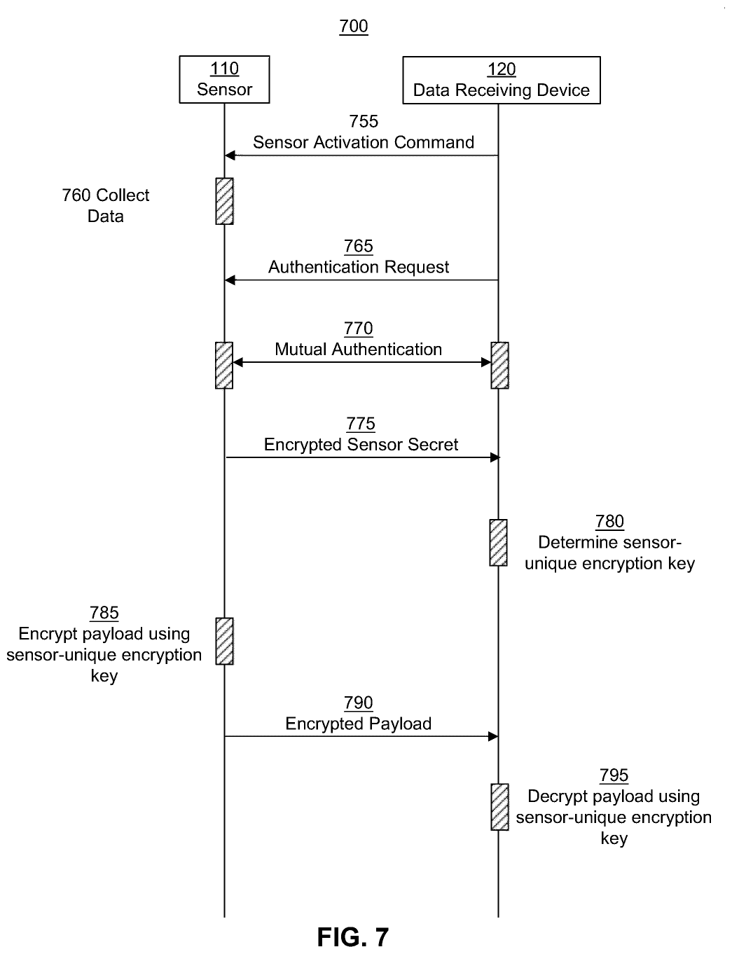

For purpose of illustration and not limitation, reference is made to the exemplary embodiment of a message sequence diagram for use with the disclosed subject matter as shown in FIG. 7 and demonstrating an example exchange of data between a pair of devices, particularly analyte sensor 110 and data receiving device 120. Data receiving device 120 can, as embodied herein, be data receiving device 120 or multi-purpose data receiving device 130. At step 755, data receiving device 120 can transmit a sensor activation command to analyte sensor 110, for example via a short-range communication protocol. Analyte sensor 110 can, prior to step 755 be in a primarily dormant state, preserving its battery until full activation is needed. After activation during step 760, analyte sensor 110 can collect data or perform other operations as appropriate to the sensing hardware 5060 of analyte sensor 110. At step 765, data receiving device 120 can an initiate authentication request command. In response to the authentication request command, both analyte sensor 110 and data receiving device 120 can engage in a mutual authentication process in step 770. Step 770 can involve the transfer of data, including challenge parameters that allow analyte sensor 110 and data receiving device 120 to ensure that the other device is sufficiently capable of adhering to an agreed-upon security framework described herein. Mutual authentication can be based on mechanisms for authentication of two or more entities to each other with or without on-line trusted third parties to verify establishment of a secret key via challenge-response. Mutual authentication can be performed using two-, three-, four-, or five-pass authentication, or similar versions thereof.

Following a successful step 770, at step 775 analyte sensor 110 can provide data receiving device 120 with a sensor secret. The sensor secret can contain sensor-unique values and be derived from random values generated during manufacture. The sensor secret can be encrypted prior to or during transmission to prevent third-parties from accessing the secret. The sensor secret can be encrypted via one or more of the keys generated by or in response to the mutual authentication process. At step 780, data receiving device 120 can derive a sensor-unique encryption key from the sensor secret. The sensor-unique encryption key can further be session-unique. As such, the sensor-unique encryption key can be determined by each device without being transmitted between analyte sensor 110 or data receiving device 120. At step 785, analyte sensor 110 can encrypt data to be included in payload. At step 790, analyte sensor 110 can transmit the encrypted payload 740 to data receiving device 120 using the communication link established between the appropriate communication models of analyte sensor 110 and data receiving device 120. At step 795, data receiving device 120 can decrypt the payload using the sensor-unique encryption key derived during step 780. Following step 795, analyte sensor 110 can deliver additional (including newly collected) data and data receiving device 120 can process the received data appropriately.

As discussed herein, analyte sensor 110 can be a device with restricted processing power, battery supply, and storage. The encryption techniques used by analyte sensor 110 (e.g., the cipher algorithm or the choice of implementation of the algorithm) can be selected based at least in part on these restrictions. Data receiving device 120 can be a more powerful device with fewer restrictions of this nature. Therefore, data receiving device 120 can employ more sophisticated, computationally intense encryption techniques, such as cipher algorithms and implementations.

### Architecture for Visualizing Impact of Personal Choices on Future Glucose Levels

The architecture described below

FIG. 8 is a block diagram of CGM system 800 that provides visualizations on the impact of personal choices on future glucose levels, according to some embodiments. CGM system 800 may include one or more of in vivo analyte sensor 104 (or in another embodiment analyte sensor 110), wearable 106, NFC source 811, data receiving device 120, trusted computer system 180, and partner API 830. Additionally, user 802 may interact with CGM system 800. CGM system 800 may be used with QR code 818.

User 802 may be a human monitoring their glucose levels using a CGM device, such as an in vivo analyte sensor (e.g., in vivo analyte sensor 104 described above). CGM system 800 may accommodate a wide-array of user types and analyte sensors. For example, user 802 may be a diabetic having type-1, type-2, or gestational diabetes. User 802 may include users across a diverse user base with different needs and interaction levels. For example, user 802 may be a proactive person with type-1 diabetes on multiple daily doses of insulin (“MDI”) that frequently checks their glucose levels, carbohydrate counts, takes post meal correction insulin boluses, and periodically titrates their insulin dosing parameter may have a good sense of how to use CGM data without additional support. However, user 802 may be a person with type-2 diabetes that does not take insulin and may be unsure why their glucose rises differently on certain occasions. User 802 may be a family member or other suitable, authorized agent of a person with diabetes. In some embodiments, user 802 may be a doctor or other medical professional. In other embodiments, user 802 may be a non-diabetic that uses the disclosed tools to monitor their health more generally. Accordingly, in addition to glucose, user 802 may monitor other analytes including ketones, lactate, oxygen, hemoglobin A1C, albumin, alcohol, alkaline phosphatase, alanine transaminase, aspartate aminotransferase, bilirubin, blood urea nitrogen, calcium, carbon dioxide, chloride, creatinine, hematocrit, lactate, magnesium, oxygen, pH, phosphorus, potassium, sodium, total protein, uric acid, etc.

Wearable 106 may be an electronic device worn by user 802. For example, wearable 106 may be a smartwatch, health-monitoring device, smart glasses or other augmented reality device, or other wearable computing device. Wearable 106 may perform health-monitoring functions, such as monitoring a user's vital signs and performing epidemiological functions (e.g., contact tracing and providing communication to an emergency medical service). Wearable 106 may also monitor physical activities of user 802, e.g., step counts and the length and intensity of exercise session and other details. In some embodiments, wearable 106 may provide additional functions, such as access to email, cellular service, and calendar functions. Wearable 106 may be worn on a user's limbs or neck, implantable in user's body, as glasses or a helmet designed to provide computer-generated reality experiences (e.g., augmented and/or virtual reality), any other suitable wearable device, and combinations thereof. Wearable 106 may be in communication with in vivo analyte sensor 104 for receiving and displaying analyte data provided by in vivo analyte sensor 104. Data receiving device 120 may also be in communication with in vivo analyte sensor 104 for receiving, processing, and/or displaying analyte data provided by in vivo analyte sensor 104.

QR code 818 may be a barcode or other coding mechanism that may be scanned by user 802 (e.g. by accessing a camera feature built into data receiving device 120). Such partner systems may include restaurants, diet programs, exercise programs, etc. For example, QR code 818 may be printed and affixed to a table in a restaurant or otherwise displayed for user 802. QR Code 818 may encode an identifier that uniquely identifies a partner restaurant, particular menu, and/or particular food choices on a menu. These food choices may include communicable sets of parameters that allow the user interface discussed below to select one or more items from a menu to create a what-if scenario predicting future glycemic information.

NFC source 811 may provide an alternative method of communicating with partner systems to receive menu options and parameters. NFC source 811 may be a hub network, peer-to-peer connections like Bluetooth, Z-Wave, ZigBee, or other appropriate configuration. NFC source 811 may rely on an appropriate NFC communication protocol to facilitate data exchange between data receiving device 120 and/or wearable 106 with partner systems.

Trusted computer system 180 is described above with reference to FIG. 1. In an embodiment, trusted computer system 180 may include servers, desktops, and other type of electronic device that support CGM system 800 by processing web-based traffic and HTTP request methods. Trusted computer system 180 may store data server-side for retrieval by data receiving device 120. CGM system may return web pages to the users via HTTP for display on display 122. The returned pages may include images, stylesheets, and scripts, the content and nature of which will be appreciated by those skilled in the relevant art(s). Trusted computer system 180 may cause data receiving device 120 to display visualizations or provide data to data receiving device 120 to generate and display of glucose-level visualizations. In an embodiment, trusted computer system 180 may include one or more of Glycemic profile emulation subsystem 821, physical activity input subsystem 822, personalized glycemic profiles 823, visualization subsystem 824, partner integration subsystem 825, app repository 826, and database 827.

Glycemic profile emulation subsystem 821 may generate a scenario that predicts future glucose levels based on different parameters including a glucose profile associated with user 802 and user input, such as food choice. Such a profile is described below as personalized glycemic profiles 1702. Glycemic profile emulation subsystem 821 may combine recent glucose history, one or more parameters associated with a metabolic model, and information related to a user choice (such as food choice or exercise) to predict the future glucose level. The one or more parameters associated with a metabolic model may be specific to a user and may be referred to as user-specific parameters. Such user-specific parameters are discussed in further detail below with reference to personalized parameters 1704. The metabolic model may be specific to a user. In one embodiment, the user-specific parameters may be retrieved from personalized glycemic profiles 823.

As described below, a food choice may also specify a quantity or portion size. The term “food” may be used interchangeably with “meal” and so food choice referred to in may also be referred to as a meal choice. Parameters may be associated with the meal choice that allow Glycemic profile emulation subsystem 821 to predict future glucose levels. Broadly speaking, parameters may be first-principles-based or clinical-practice-based. In the context of meal choice, first-principles-based parameters and clinical-practice-based parameters correspond to parameters that are involved in describing the effect of a meal on a person's glucose, either based on first-principles derivation or clinical practice. For example, first-principles-based models are generally represented by one or more equations with variables that vary over time, representing physical or conceptual compartments involved in the process of a meal affecting the person's glucose. The parameters involved in these equations are considered first-principles-based parameters and predominantly involve compartment-based-modeling—i.e., modeling one or more compartments with a pre-determined static or dynamic relationship amongst them. In contrast, clinical-based-parameters are parameters that are commonly used to describe certain quantification and/or qualification of the nature of the meal effect on glucose in clinical practice. However, there is no hard boundary between these distinctions. For example, first-principles-based parameters specific to a meal choice may be: (1) rate of glucose appearance profile in an averaged person with standard food bioavailability, and (2) information needed for a user's gastric emptying related to the meal choice such as solid-liquid ratio of the meal. Examples of clinical-practice-based parameters specific to a meal choice may be: (1) glycemic index, (2) glycemic load, (3) macronutrient composition (percentage of carbohydrate, fat and protein), and (4) fiber content.

Because a meal choice may use first-principles-based parameters that are more naturally mathematically modelled, Glycemic profile emulation subsystem 821 may support differing classes of meal information. For example, categorization fields may be the branded name of a food item as chosen by a partner restaurant (e.g., a blooming onion at the OUTBACK STEAKHOUSE®), generic meal name (e.g. whole fried onion), or a general category (e.g. fried appetizer). To facilitate this duality, Glycemic profile emulation subsystem 821 may generate a random forest module or other supervised machine learning model that reconciles all possible combinations of meal information types to provide unified meal input information. Such models are described below as pre-trained models 1701.

Alternatively, Glycemic profile emulation subsystem 821 may pass different combinations of meal-input information to different glycemic profile emulation sub-modules. Glycemic profile emulation subsystem 821 may then rely on a pre-defined, gain-scheduling approach placed downstream that weighs all possible sub-modules and generates a single emulated glycemic profile.

Alternatively, Glycemic profile emulation subsystem 821 may employ a compartment-modelling-based approach to describe the effect from different meal input information such as carbohydrate content, glycemic index, food composition, order of food consumption and so on, with the output being the dynamic profile of post meal glycemic excursions where some key population model parameters may be estimated. The key population model parameters may include time-of-maximum appearance rate of glucose, carbohydrate bioavailability, gut hormone secretion, plasma glucose pool size, endogenous glucose production, utilization of glucose, beta-cell sensitivity and insulin sensitivity, etc.

Physical activity input subsystem 822 may emulate the effect of physical activity. The effect of physical activity on a blood glucose level may vary depending on many factors including the mode of exercise, duration, and intensity. In general, most forms of aerobic/cardiovascular exercise can lower blood glucose for up to 24 hours or more during and/or after working out by making the body more sensitive to insulin. However, blood glucose may go up after certain intensive physical activity such as heavy weightlifting, sprints, and competitive sports due to rising levels of stress hormones (e.g., adrenaline). Previous research shows that exercise may cause profound changes in glucose homeostasis, especially in people with diabetes. In healthy individuals, acute moderate exercise (at 50% VO² max) almost doubles postprandial insulin sensitivity index. The impact of physical activity on substrate utilization, including glucose utilization, may also be sex-dependent.

Depending on the richness of data communicated by physical activity input subsystem 822, an exercise session may be handled similarly to meal input. Data of interest for the physical activity input subsystem 822 may include parameters that allow the model to link the impact of physical activity to other measurable metrics that wearable 106 may measure (e.g., heart rate, steps, elevation change, etc.). Examples of data associated with physical activity include endogenous glucose production (“EGP”) and glucose utilization related parameters, such as stress hormone levels and insulin sensitivity, as a function of intensity and duration, exercise mode, and demographics information. The post exercise emulated glycemic profiles may be obtained through a similar approach as for the post-meal emulated glycemic profiles. Alternatively, the effect of physical activity may be modelled as a separate component to be added into the aforementioned meal intake compartment model through key physical activity parameters such as intensity, duration, and mode of exercise. To provide one example of a meal intake compartment model, the meal absorption model may be represented by two compartments and defined by the following equations:

$$\frac{d {a}_{1} ( t )}{d t} = - \frac{1}{{t}_{max , G}} {a}_{1} ( t ) + {δ}_{tj} ( t ) {u}_{G} ( tj )$$

$$\frac{d {a}_{2} ( t )}{d t} = \frac{1}{{t}_{max , G}} ( {a}_{1} ( t ) - {a}_{2} ( t ) )$$

$${U}_{M} ( t ) = \frac{5.5 \; 56 \cdot {A}_{G} {a}_{2} ( t )}{{t}_{max , G} {V}_{G} W}$$

In these equations, a₁(t) and a₂(t) represent carbohydrate amount in the first and second meal absorption compartment, respectively (g). The parameter u_{G}(tj) represents the carbohydrate amount eaten at time tj(g), t_{max,G} is the time-of-maximum appearance rate of glucose (min), AG is the fractional bioavailability (unitless), VG is the plasma glucose pool size (1/kg), and UM (t) is the gut carbohydrate absorption rate with unit converted to glucose concentration rate of change (mmol/l/min).

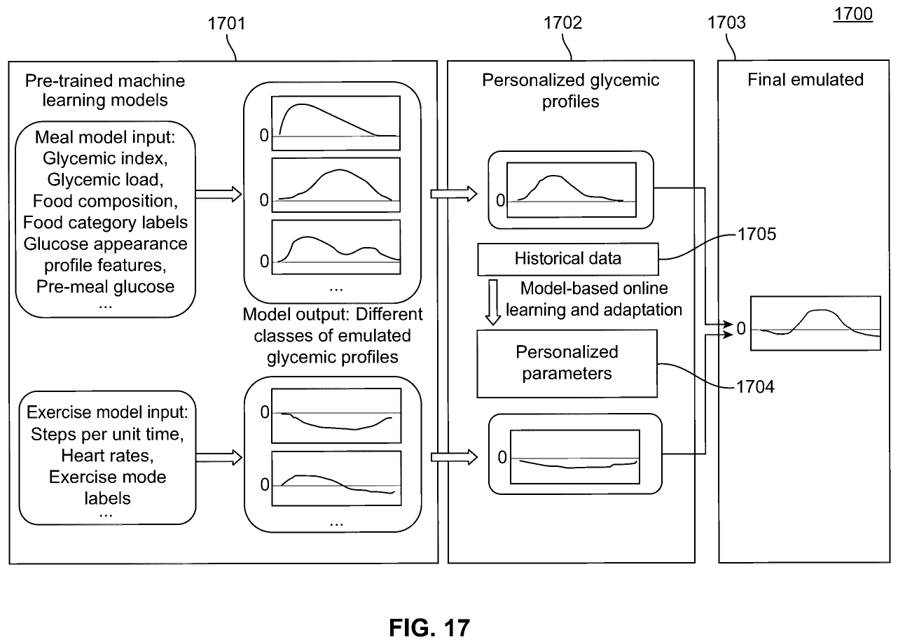

Personalized glycemic profiles 823 may include historical data and personalized parameters for a particular user 802. Historical data may include past readings of glucose data tracked over time and data and parameters about past and/or current exercise sessions. Personalized glycemic profiles 823 may also include personalized adjustment parameters, such as macronutrients bioavailability, and insulin sensitivity. Personalized glycemic profiles 823 may also include user-specific parameters. The user-specific parameters may comprise personalized adjustment parameters that are discussed in further detail below with reference to personalized parameters 1704. This allows maximum compatibility with meal and physical activity related information provided by partner systems, and allows Glycemic profile emulation subsystem 821 to adapt to a user's metabolic condition over time. Personalized glycemic profiles 823 are detailed further below with reference to FIG. 17.

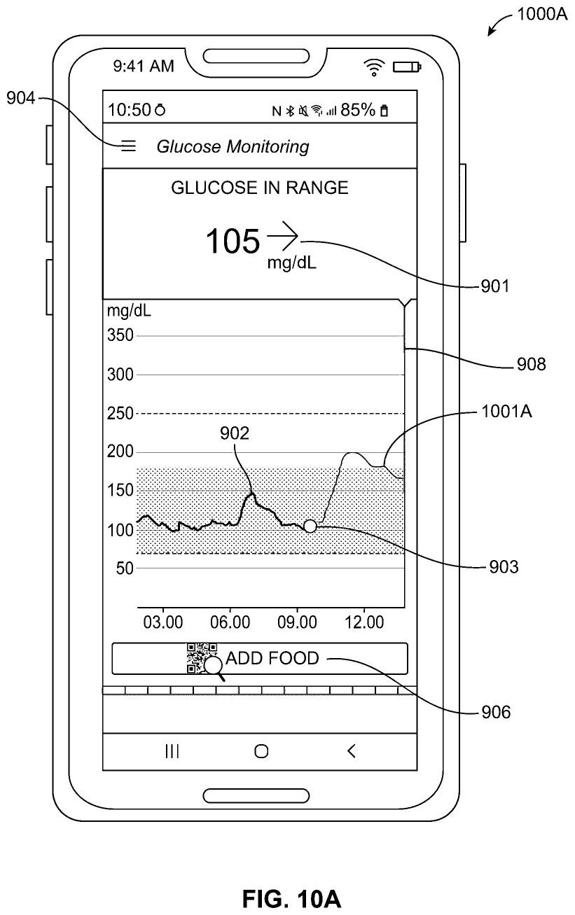

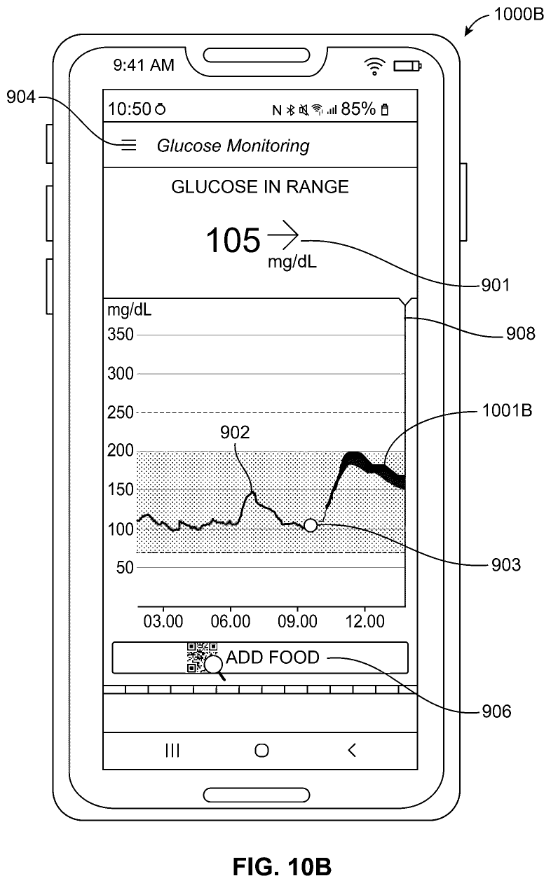

Visualization subsystem 824 may generate PIE visualizations. Such a “butterfly effect” visualization allows user 802 to visualize the impact of personal choices on estimated future glucose data. Visualization subsystem 824 may present future data as a single trace (i.e., a line), a range, or using another suitable approach to make the consequences of choices personally relatable to user 802 in terms of their near future glycemic profile. Examples of visualizations generated by visualization subsystem 824 are discussed below with reference to FIGS. 10A-B, 11A-B, and 12A-B.

Partner integration subsystem 825 may integrate with partner systems. Such partner systems may include restaurants, diet programs, exercise programs, etc. Partner integration subsystem 825 may access APIs provided by partner system to receive appropriately structured and parameterized information about food choices and/or exercise sessions. As discussed below, this parameterized information may be used by Glycemic profile emulation subsystem 821 to predict future glucose values.

App repository 826 may be a catalog of installable applications from which client-side components running on data receiving device 120 may be downloaded and installed. While FIG. 8 displays app repository 826 within trusted computer system 180, in other embodiments, app repository 826 may be a widely used app repository from which mobile device applications may be downloaded and installed onto data receiving device 120.

Database 827 may store information about glucose monitoring, past readings, configuration, user information, partner information, and other information relevant to CGM system 800. Database 827 may be a relational database, a NoSQL database or other horizontally scaling database, a digital ledger technology or blockchain, or any other suitable storage mechanism. For instance, database 827 may be a commercially available database management system to store and retrieve data. In an embodiment, database 827 may be retrieved data from a centralized storage area network (SAN), network-attached storage (NAS), redundant array of independent disks, and/or any other configuration of storage devices to supply sufficient storage capacity to store database tables and supporting structures. Sufficient storage may alternatively exist in any other physically attached magnetic storage, cloud storage, or additional storage medium. In an embodiment, database 827 may deploy a hard-disk interface, such as ATA, SATA, SCSI, SAS, and/or fibre for interfacing with storage mediums housing data.

Partner API 830 may be a secure interface that facilitates interactions between data receiving device 120 and partner systems. Such partner systems may include restaurants and diet programs. Partner API 830 is associated with an endpoint, i.e., a resource (often represented as a unique URL) that may accept requests to the services provided. For example, Partner API 830 may leverage various communication standards and protocols, e.g., TLS, SSL, HTTP, HTTPS, etc., to further communication between various components. Partner API 830 may provide an addition level of security for both the client/requestor and server/responder because limited types of communications transpire between the client and server, obviating the need for any party to fully expose its data. By accepting and responding to requests, partner API 830 may provide functions that allow data receiving device 120 to receive a particular menu and/or particular food choices on a menu from a partner restaurant. These food choices may include communicable sets of parameters that allow the user interface discussed below to select one or more items from a menu to create a what-if scenario predicting future glycemic information.

### PIE Visualization User Interfaces

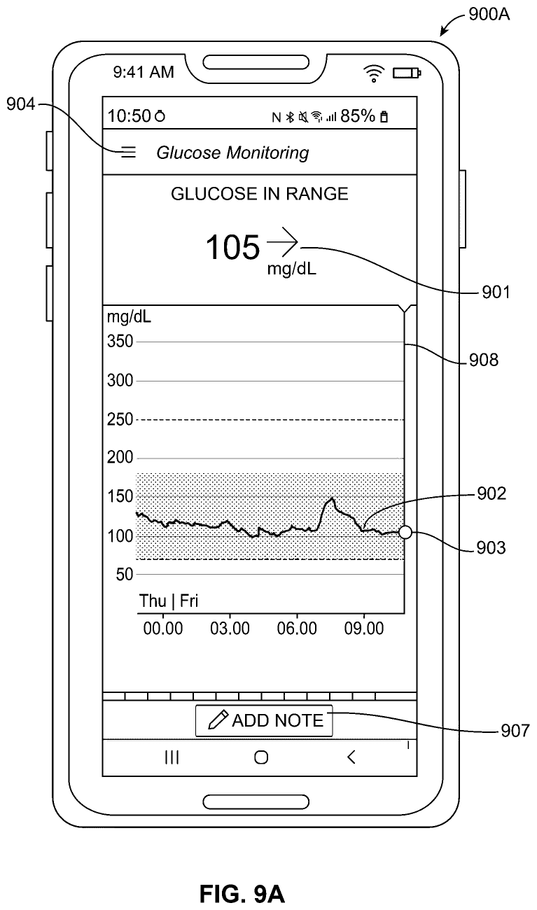

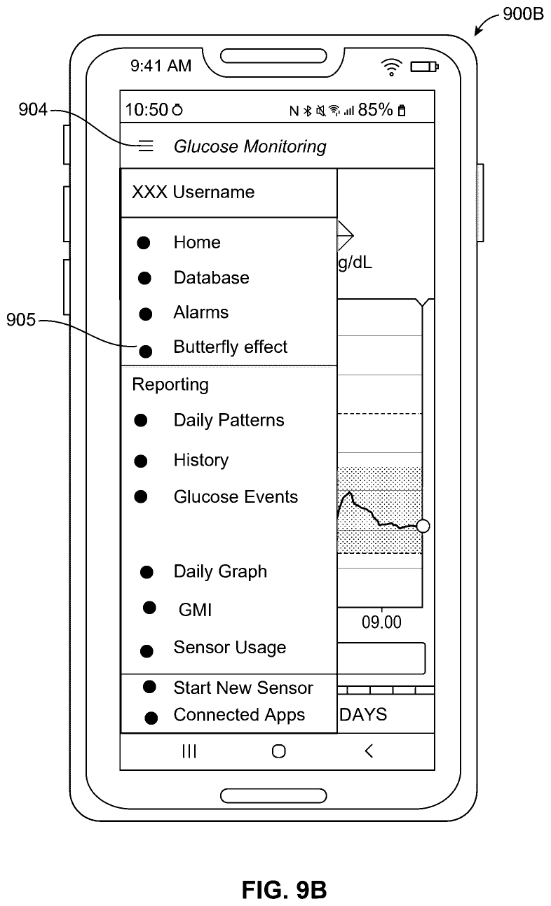

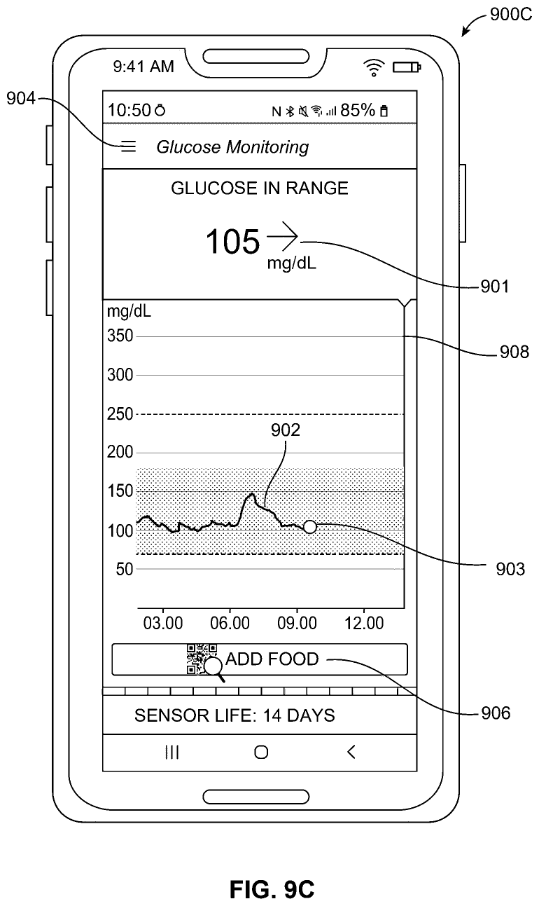

FIG. 9A is an example hybrid sensor-meal visualization 900A of a CGM user interface that provides a visualization of past blood glucose levels, according to some embodiments. The screen display provided in FIG. 9A is merely exemplary, and one skilled in the relevant art(s) will appreciate that many approaches may be taken to provide a hybrid sensor-meal visualization 900A in accordance with this disclosure.

Dynamic visualization component 901 comprising a current glucose reading may display the most recent glucose measurement received by data receiving device 120 from in vivo analyte sensor 104. In the example in FIG. 9A, this value is 105 mg/dL, but one skilled in the relevant arts will appreciate that this value will vary over time and also that other measurements may be used to track glucose levels that are also applicable within the context of this disclosure. In some embodiments, dynamic visualization component 901 comprising a current glucose reading may further include a timestamp that the reading was taken and/or additional biometric data such as heart rate, respiration rate, etc.

Hybrid sensor-meal visualization 900A further comprises glucose visualization component 908 configured to display glucose data over a predefined and configurable period of time (e.g., 3 hours, 9 hours, 12 hours). Glucose visualization component 908 may include past readings 902 which reflect continuous readings of glucose levels taken over time by in vivo analyte sensor 104. Past readings 902 is a line graph in FIG. 9A, but other visualizations may be suitable for displaying past readings 902 as well. Moreover, while the time-scale in FIG. 9A is a single day, additional functionality may be provided to allow a user to zoom out or zoom in by widening or narrowing the time window. Glucose visualization component 908 may further include a recent time 903 may be a point in time reflecting the most recent received reading. In some embodiments, recent time 903 may reflect a current time in addition to a point on the visualization. In some embodiments, recent time 903 may reflect the latest time for the glucose data.

Menu button 904 may be a navigation icon that user 802 may select to encounter more options in the user interface. One possible menu is displayed below in FIG. 3B that may display in response to user 802 clicking or tapping on menu button 904. Note visualization component 907 may allow user 802 to add a note in association with a past reading or dynamic visualization component 901 comprising a current glucose reading.

FIG. 9B is an example hybrid sensor-meal visualization 900B of a CGM user interface including a menu of options, according to some embodiments. The screen display provided in FIG. 9B is merely exemplary, and one skilled in the relevant art(s) will appreciate that many approaches may be taken to provide hybrid sensor-meal visualization 900B in accordance with this disclosure. Hybrid sensor-meal visualization 900B may display upon user 802 selecting menu button 904.

Butterfly effect option 905 may a selectable option in the displayed menu. Butterfly effect option 905 may be a navigational link, button, hyperlink, or other navigational construct that routes a user to a page that allows user 802 to visualize the “butterfly effect” of future decisions. Because diet and exercise choices may lead to changes in their glucose levels at a later time, user 802 may benefit from understanding the “butterfly effect” of diet and exercise choices. Upon clicking or otherwise engaging butterfly effect option 905, user 802 may be routed to the butterfly effect page, which is displayed in FIG. 9C.

FIG. 9C is an example hybrid sensor-meal visualization 900C of a CGM user interface including an updated glucose visualization component 908 to include past blood glucose levels and future times, according to some embodiments. Glucose visualization component 908 provided in FIG. 9C is merely exemplary, and one skilled in the relevant art(s) will appreciate that many approaches may be taken to provide a glucose visualization component 908 in accordance with this disclosure. Glucose visualization component 908 may include a note visualization component 907 for receiving user input related to food choices in response to a selection of butterfly effect option 905. Note visualization component 906 can enable the user to provide input to a machine learning model for updating the visualization components and generating a predicted glucose impact based on available data. Examples of note visualization component 906 include food/meal options and activities (such as exercise), which can be provided as input to a trained machine learning model for generating the predicted glucose values and, in some embodiments, the associated visualization components for the hybrid sensor-meal visualization.

In one embodiment, a model implemented within glycemic profile emulation subsystem 821 may be used to calculate predicted glycemic insights, which are then represented visually on a graph. The model can make the predictions at regular intervals, for instance, every few minutes, with the visualization connecting the predicted values through a smoothed line that reflects these discrete calculations. This approach offers a stepwise prediction where the line is drawn based on individual predicted values over time. Glycemic profile emulation subsystem 821 is configured to communicate with in vivo analyte sensor 104, and the intervals between the predictions is determined based on the interval of data provided by in vivo analyte sensor 104.

Alternatively, the model could predict how glucose levels are expected to change continuously over time, rather than providing discrete values. In this case, the sloped line in the graph would represent the anticipated trajectory of glucose change, calculated using continuous data rather than separate points. In this embodiment, the model generates a dynamic representation of glucose changes. The sloped line can represent the anticipated trajectory of glucose levels based on these continuous predictions, providing another view of expected trends and allowing users to understand how their glucose levels may change in response to various factors in real time.

Glycemic profile emulation subsystem 821 can be configured to adjust the type of prediction (e.g., stepwise or continuous) based on one or more of the timing of available glucose data, user preference, and whether glycemic profile emulation subsystem 821 has communicated with in vivo analyte sensor 104 within a predetermined amount of time.

In some embodiments, glucose visualization component 908 may include a time axis is relative to an event (e.g., a recent time, a current time) and user provided input (e.g., exercise, meal, etc.), allowing users to understand how their glucose levels respond before, during, and after the event based on the user provided input. In some embodiments, glucose visualization component 908 is configured to provide a graph that depicts sensor glucose levels as part of a glycemic insight message, in relation to a predicted glycemic impact. Glucose visualization component 908 can configure the time axis to show measured glucose levels relative to an event, such as a current time, and a user provided activity, such as physical activity or a meal. In this visualization, one line could represent the measured glucose levels (e.g., provided by in vivo analyte sensor 104), while another line can represent a predicted glycemic impact.

Note visualization component 906 may allow user 802 to add a particular food choice and immediately visualize the impact of the choice on their future glucose levels. For example, user 802 may contemplate what to cat for dinner at a partner restaurant. User 802 may see note visualization component 906 in hybrid sensor-meal visualization 900C and click or tap note visualization component 906. In one embodiment, user 802 may select from a list of general food options, select portion sizes, etc. In another embodiment, user 802 may select from menu options provided by the partner system in response to scanning QR code 818 or receiving menu options via NFC source 811. The food menu choices may include communicable set of parameters for each choice. Parameters received for each menu choice may include one or more of: (1) rate of glucose appearance profile; (2) gastric emptying information; (3) glycemic index; (4) glycemic load; (5) macronutrient composition (percentage of carbohydrate, fat and protein); and (6) fiber content.

FIG. 10A is an example hybrid sensor-meal visualization 1000A of a CGM user interface including glucose visualization component 908 updated to display single trace of estimated future glucose levels based on a food selection, according to some embodiments. The screen display provided in FIG. 10A is merely exemplary, and one skilled in the relevant art(s) will appreciate that many approaches may be taken to provide a hybrid sensor-meal visualization 1000A in accordance with this disclosure.

Predicted glucose 1001A may be displayed in response to user 802 selecting a food option as discussed above with reference to FIG. 9C. Predicted glucose 1001A may display as a line graph or other suitable visualization. In the example provided in FIG. 10A, predicted glucose displays as a line starting at recent time 903 and proceeding several hours into the future. Other suitable time ranges may be employed. Predicted glucose 1001A may be displayed in continuity with past readings 902 with respect to recent time 903 such that the transition at recent time 903 between past glucose readings and future glucose readings is graphically seamless. In some embodiments, predicted glucose 1001A may be visually distinguished from past readings 902 such that predicted glucose 1001A and past readings 902, such via different colors, shading, and lines (e.g., varying thicknesses, colors, and line patterns). In some embodiments, predicted glucose 1001A may be displayed as an overlay over the past readings 902.

In some embodiments, glucose visualization component 908 may dynamically adjust visualization of predicted glucose 1001A based on one or more of the available glucose levels, a confidence score of the predicted glucose 1001A, and available sources that provided data for the predicted glucose 1001A. Glycemic profile emulation subsystem 821 can include the confidence score as part of its predictions. The confidence score can be considered a measure of the reliability of the predicted values and can based on several factors. For example, glycemic profile emulation subsystem 821 can factor account the available glucose data; when there is less reliable data, such as missing or inconsistent readings, the confidence score will be lower. As another example, glycemic profile emulation subsystem 821 factors the number of input sources (e.g., the amount of glucose data, the amount of user historical data such as their eating history, glycemic impact history) being used-having fewer input sources leads to a lower confidence score, while multiple reliable input sources can increase the score. Finally, glycemic profile emulation subsystem 821 can also consider the last time the in vivo analyte sensor 104 communicated with the visualization component. For example, if the data is outdated or there is a significant delay in communication, the confidence score will decrease, reflecting the reduced reliability of the prediction.

FIG. 10B is an example hybrid sensor-meal visualization 1000B of a CGM user interface including glucose visualization component 908 updated to display of ranges of estimated future glucose levels based on a food selection, according to some embodiments. In some examples, the UI displayed in FIG. 10B may be generated by visualization subsystem 824 of FIG. 8. The screen display provided in FIG. 10B is merely exemplary, and one skilled in the relevant art(s) will appreciate that many approaches may be taken to provide hybrid sensor-meal visualization 1000B in accordance with this disclosure.

As in FIG. 10A, predicted glucose 1001B may be displayed in response to user 802 selecting a food option. In FIG. 10B, predicted glucose 1001B displays as a range of possible values. The range of possible values may bounded by a predicted minimum value and a predicted maximum value. In some examples, the UI displayed in FIG. 10B may be generated by visualization subsystem 824 of FIG. 8.

In some embodiments, hybrid sensor-meal visualization 1000B is configured to adjust visual properties of predicted glucose 1001B to emphasize the range of values between the upper and lower bounded lines. For example, predicted glucose 1001B may be represented by a shaded region between upper and lower bounded lines.

In some embodiments, the visual properties may be based on a confidence metric associated with predicted glucose 1001B. The confidence metric may reflect a likelihood of accuracy of predicted glucose 1001B as determined by visualization subsystem 824. As a non-limiting example, the confidence metric may be based on the glucose data provided to visualization subsystem 824, which may give more weight to more recent glucose data compared to glucose data with timestamps older than a predetermined period (e.g., 15 minutes, 30 minutes, 1 hour). As another example, the confidence metric may be based on data that is available to visualization subsystem 824 when generating the predicted glucose 1001B.

In some embodiments, the range of possible values is graphically juxtaposed in relation to the recent time 903 and can be visually distinguished (e.g., by color, gradation, indicators) from the past readings 902.

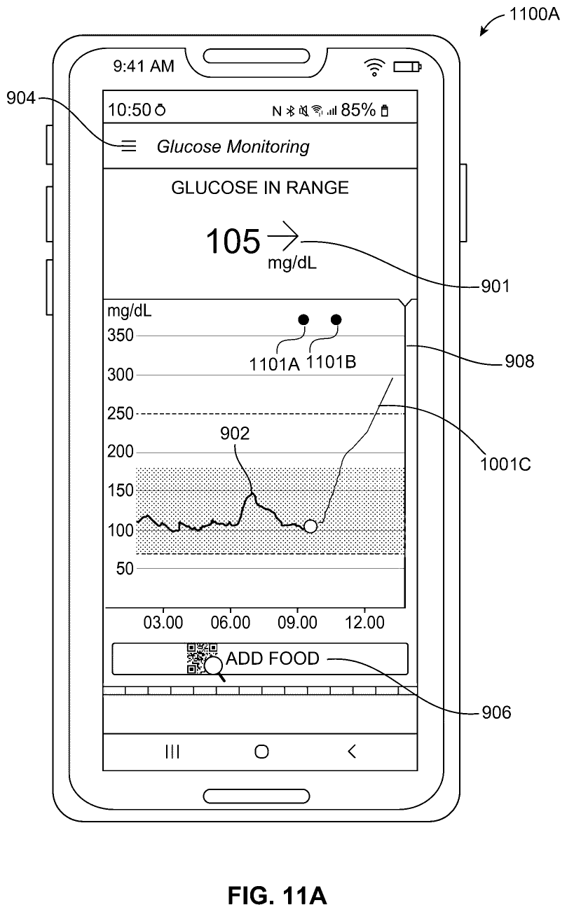

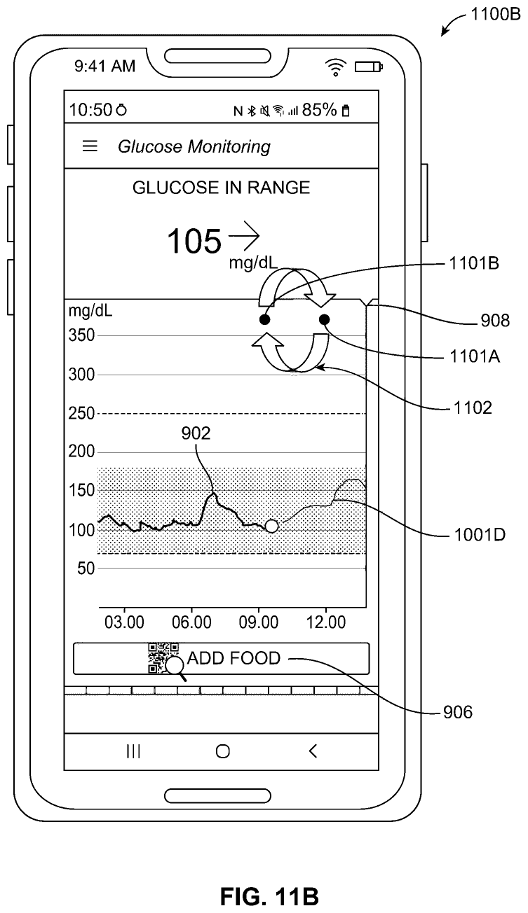

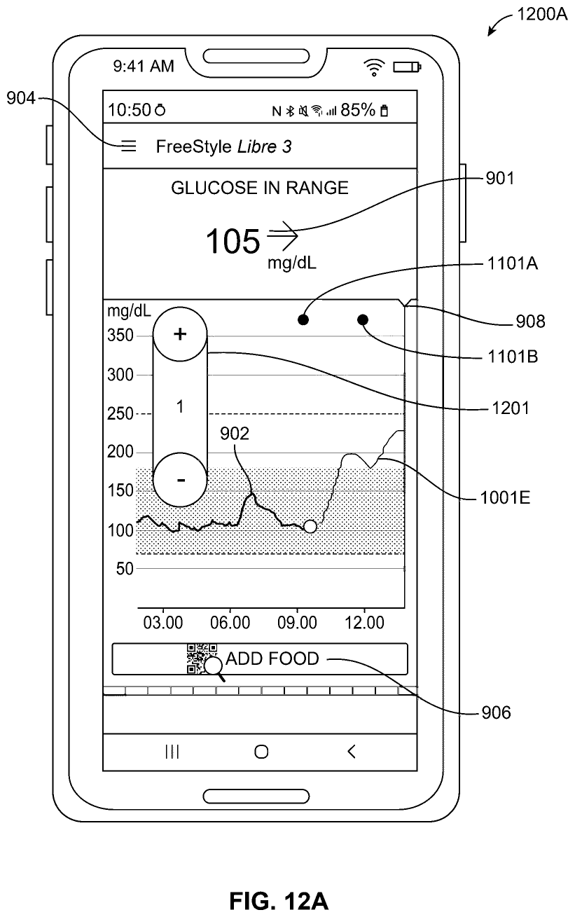

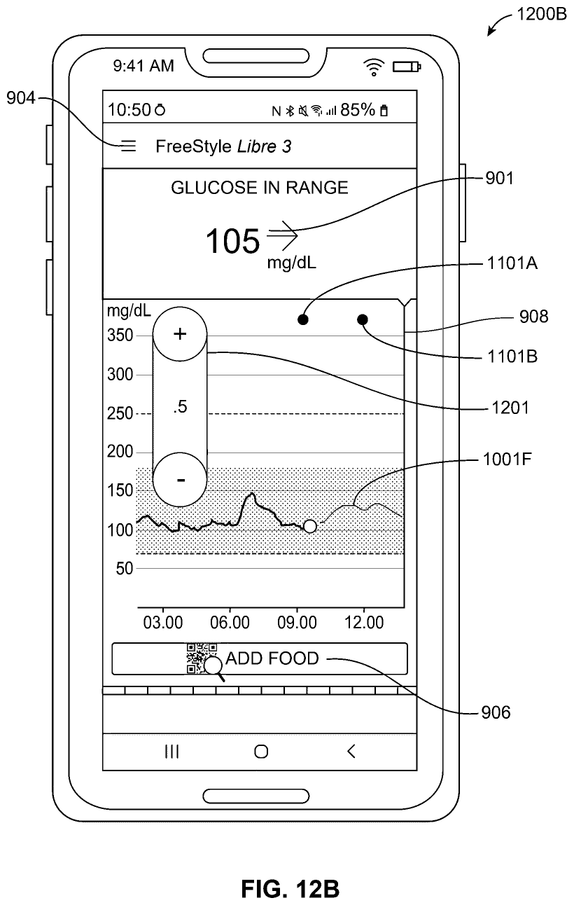

FIGS. 11A, 11B, 12A, and 12B provide exemplary visualizations of estimated future glucose levels that provide glucose visualization component 908 for enabling real-time adjustment of inputs for updating predicted glucose impacts. For example, each visualization may display interactive visual objects (e.g., meal real-time user input component 1101A or real-time portion selector 1201) for receiving user adjustments to meal sequences or portions and the user interface is configured to dynamically update the estimated future glucose levels by communicating the provided adjustments to a machine learning model which provides updated estimated future glucose levels. The CGM user interface for FIGS. 11A, 11B, 12A, and 12B may then be configured to dynamically update the visualization of the estimated future glucose levels in real-time in response to the user adjustments received via the

FIG. 11A is an example hybrid sensor-meal visualization 1100A of a CGM user interface including glucose visualization component 908 updated to display estimated future glucose levels based on multiple food selections, according to some embodiments. The screen display provided in FIG. 11A is merely exemplary, and one skilled in the relevant art(s) will appreciate that many approaches may be taken to provide a hybrid sensor-meal visualization 1100A in accordance with this disclosure.

Real-time user input component 1101A and real-time user input component 1101B may be configured to adjust food choices provided via visualization component 906. By including these interactive visual objects in the visualization, user 802 may update the events that factor into the estimated future glucose levels. For example, as discussed below, user 802 may control the portion sizes of these meal choices. User 802 may also edit the meal choices to change the added food and/or delete the meal choice entirely. For example, user 802 may click, tap, or hold real-time user input component 1101A and receive a pop-up menu that allows user 802 to edit the information associated with real-time user input component 1101A. One or both of these events may alternatively be exercise sessions—i.e., either of the dots representing real-time user input component 1101A and real-time user input component 1101B may represent added exercise sessions. In that sense, Glycemic profile emulation subsystem 821 may generate a future predicted glucose level that considers the impact of both a meal choice and/or an exercise session. In FIG. 11A, predicted glucose 1001C is displayed as rising first in response to real-time user input component 1101A and then again in response to real-time user input component 1101B.

FIG. 11B is an example hybrid sensor-meal visualization 1100B of a CGM user interface including glucose visualization component 908 updated to display estimated future glucose levels based on multiple food selections with a sequencing option, according to some embodiments. The screen display provided in FIG. 11B is merely exemplary, and one skilled in the relevant art(s) will appreciate that many approaches may be taken to provide hybrid sensor-meal visualization 1100B in accordance with this disclosure.

Sequence selector 1102 may allow user 802 to edit the sequencing of food consumption and/or exercise. For example, if a user has added a first food option of mixed green salad and a second food option of tiramisu, sequence selector 1102 may allow user 802 to toggle whether the green salad is eaten before or after the tiramisu. In one embodiment, sequence selector 1102 may swap real-time user input component 1101A and real-time user input component 1101B with a single click or tap. In this fashion, sequence selector 1102 allows user 802 to control a temporal ordering of the meal choices to determine which is eaten first, second, third, etc. In another embodiment, user 802 may drag real-time user input component 1101B to a location before/after real-time user input component 1101A. For example, user 802 may click or tap and drag the food icon to reorder as shown on the right panel of FIG. 11B (e.g., drag real-time user input component 1101A to the right and the system would swap its position with real-time user input component 1101B as shown, or drag real-time user input component 1101B to the left and the system would its position with real-time user input component 1101A as shown). In response to the latest selection (e.g., with or without further user input), Glycemic profile emulation subsystem 821 may recalculate a predicted near future glucose impact and visualization subsystem 824 may automatically overlay the updated predicted glucose information in the visualization. In this example, predicted glucose 1001D has lowered based on the updated sequencing as compared to predicted glucose 1001C.

FIG. 12A is an example hybrid sensor-meal visualization 1200A of a CGM user interface including glucose visualization component 908 updated to display estimated future glucose levels based on multiple food selections with a portion controller, according to some embodiments. The screen display provided in FIG. 12A is merely exemplary, and one skilled in the relevant art(s) will appreciate that many approaches may be taken to provide sensor-meal visualization 1200A in accordance with this disclosure.

Real-time portion selector 1201 may provide user 802 a convenient way of allowing user 802 to explore the impact of reducing or increasing the portion size of a meal choice. In some examples, real-time portion selector 1201 appears (e.g., is displayed) when the user clicks/taps on a meal choice. Real-time portion selector 1201 may allow user 802 to tap on the food icon and then either drag the icon in the up/down direction or by using an adjustment bar. In an alternative embodiment, merely dragging real-time user input component 1101A down in the visualization may reduce the portion size. Conversely, dragging real-time user input component 1101A up may increase the portion size. In this example, predicted glucose 1001E is spiking above 200 mg/dL.

FIG. 12B is an example hybrid sensor-meal visualization 1200B of a CGM user interface including glucose visualization component 908 updated to display estimated future glucose levels based on multiple food selections with a movable portion controller, according to some embodiments. The screen display provided in FIG. 12B is merely exemplary, and one skilled in the relevant art(s) will appreciate that many approaches may be taken to provide a sensor-meal visualization 1200B in accordance with this disclosure.

Altering the timing of meal choices, quantity (e.g., eating half of the portion), items, or order of consumption can result in an updated prediction of the near future glucose impact. For example, predicted glucose 1001F of FIG. 12B has lowered as compared to predicted glucose 1001E of FIG. 12A in light of the smaller portion selected in FIG. 12B.

### Impact of Physical Activities

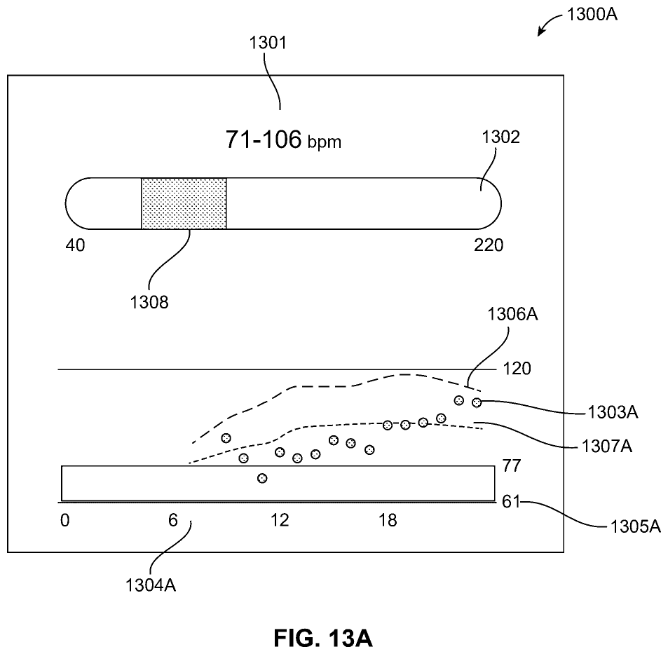

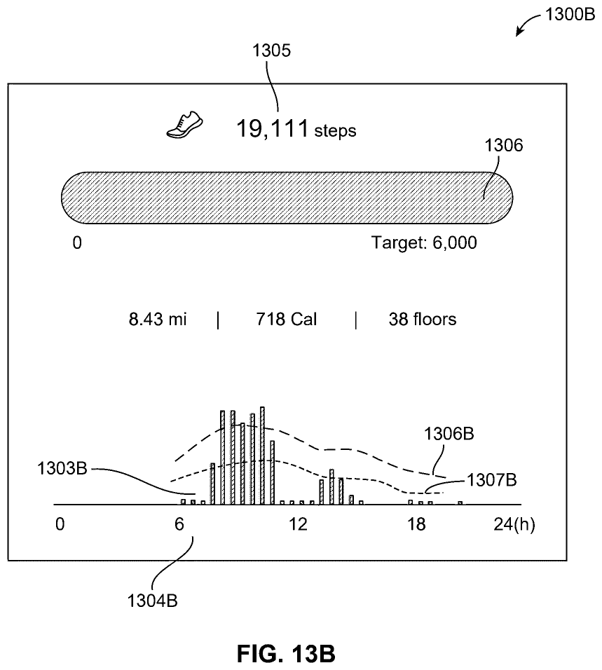

FIG. 13A is an example hybrid sensor-meal visualization 1300A of a CGM user interface that displays heart rate and glucose levels, according to some embodiments. The screen display provided in FIG. 13A is merely exemplary, and one skilled in the relevant art(s) will appreciate that many approaches may be taken to provide hybrid sensor-meal visualization 1300A in accordance with this disclosure.

Beats per minute (“BPM”) range 1301 may display the minimum and maximum heart rates in BPM over a time period/time range. In the exemplary illustration in FIG. 13A, the time range is 24 hours, but other suitable time ranges may be employed. In one embodiment, user 802 may update range 1301 (e.g., by dragging to the right or left) and the projected ranges in the visualization update as the user moves the range to the right or left or after the user releases the sliding scale.

BPM indicator 1302 may indicate where BPM range 1301 falls on a continuum of heart rates. In this example, the continuum runs from 40 BPM to 220 BPM. BPM indicator 1302 provides an indication of where the BPM data falls relative to normal rates among the general population. Moreover, an ideal or desired range may be indicated by range 1308. Range 1308 may default to particular values.

Glucose data visualization component 1303A can be configured to display past readings of a particular data point for user 802 received from in vivo analyte sensor 104, readings of heart rate and other suitable physiological readings, a number of steps, number of pushes (e.g., users in a wheel chair), or any other data tracked by wearable 106. In the illustrative example in hybrid sensor-meal visualization 1300A, glucose data visualization component display heart rate readings, as captured in the last 24 hours by wearable 106.

X-axis 1304A may represent a time frame. In the example provided in hybrid sensor-meal visualization 1300A, X-axis 1304A displays the past 24 hours. However, other time frames may be appropriate. In some embodiments, user 802 may change and/or configure this time frame. Moreover, in some embodiments, future times may be included on x-axis 1304A and predicted glucose levels displayed—e.g., x-axis 1304A may include the past 12 hours and the future 12 hours. In another embodiment, only future times may display on x-axis 1304A.

Y-axis 1305A may display a range of readings corresponding to glucose data visualization component. In this illustrative example, hybrid sensor-meal visualization 1300A displays a range from 61 to 120 bpm, with 61 to 77 bpm indicated as the target range. As discussed above, range 1308 may be updated by a user, and in such an instance, Y-axis 1305A may update accordingly.

Upper range curve 1306A may range reflect an upper bound of recorded and estimated glucose levels for user 802 received from in vivo analyte sensor 104. Lower range curve 1307A may be a lower bound of the recorded and estimated glucose levels for user 802. These upper and lower bounds allow user 802 to visualize the continuation of their present glucose through the duration of their physical activity. Additionally, in an embodiment, upper range curve 1306A and lower range curve 1307A may extend into the future to display the future glucose levels estimated by Glycemic profile emulation subsystem 821.

FIG. 13B is an example hybrid sensor-meal visualization 1300B of a CGM user interface that displays step counts and glucose levels, according to some embodiments. The screen display provided in FIG. 13B is merely exemplary, and one skilled in the relevant art(s) will appreciate that many approaches may be taken to provide hybrid sensor-meal visualization 1300B in accordance with this disclosure.

Number of steps 1305 may be a number of steps recorded by wearable 106. Steps goal 1306 may be target number of steps that varies over time based on a level of activity of user 802. Depending on an intended physical activity choice, the lower and upper bounds may allow user 802 to visualize the continuation of their present glucose through the duration of their physical activity.

In one embodiment, the user interface may allow user 802 to adjust the duration and intensity of a future or past exercise and view the results in an updated projection of the user's near future glycemic profile. For example, by interfacing with wearable 106 of FIG. 8, physical activity input subsystem 822 may also provide measurable ranges associated with the selected activity, e.g., number of steps, number of pushes, distance covered, a target time course of the heart rate, etc. This allows user 802 to ensure that their chosen physical activity follows the projection. The upper and lower ranges provide a general indication on whether the resulting post-exercise glucose will be lower or higher than the predicted near future glucose prior to the start of the physical activity.

Recorded levels 1303B may represent past readings of a particular data point. In the illustrative example in hybrid sensor-meal visualization 1300B, recorded levels 1303 display the number of steps in increments, as captured in the last 24 hours by wearable 106. X-axis 1304B may represent a time frame. In the example provided in hybrid sensor-meal visualization 1300B, X-axis 1304B displays the past 24 hours. In some embodiments, future times may be included on x-axis 1304B and predicted glucose levels displayed—e.g., x-axis 1304B may include the past 12 hours and the future 12 hours. In another embodiment, only future times may display on x-axis 1304B.

Upper range curve 1306B may range reflect an upper bound of recorded glucose levels for user 802 received from in vivo analyte sensor 104. Lower range curve 1307B may be a lower bound of the recorded glucose levels for user 802. These upper and lower bounds allow user 802 to visualize the continuation of their present glucose through the duration of their physical activity. Additionally, in an embodiment, upper range curve 1306B and lower range curve 1307B may extend into the future to display the future glucose levels estimated by Glycemic profile emulation subsystem 821. Moreover, a hybrid sensor-meal visualization may display how their glucose levels may change if they complete additional steps. For example, a user may update the target from 6,000 steps to 10,000 and view the impact upon predicted further glucose levels as reflected in upper range curve 1306B and lower range curve 1307B.

In addition to fusing and/or integrating the glucose for user 802 into a model (e.g., a machine learning model) that projects glucose as a result of one type of choice, a combination of choices (e.g. exercise before vs after the meal) may be integrated to produce a combined effect. As certain macronutrients such as the persistent selection of high fat foods and regular moderate physical activity can impact a metabolic parameter, Glycemic profile emulation subsystem 821 may also take this historic trajectory of choices into account. A different type of what-if scenario may be provided to encourage the user to maintain the combination of healthy choices. One example is to provide a projected post-meal glucose if the user stops regular physical activity in the near future.

### Personalized Glycemic Profile Emulation Module

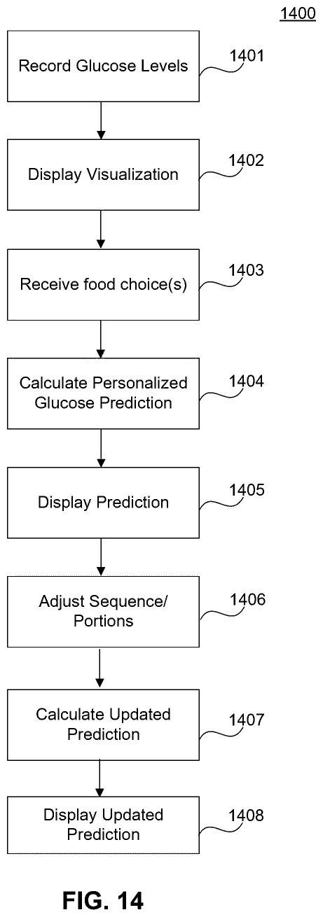

FIG. 14 is a flowchart illustrating a method 1400 of providing a personalized glucose prediction based on a food choice, according to some embodiments. Method 1400 can be performed by processing logic that can comprise hardware (e.g., circuitry, dedicated logic, programmable logic, microcode, etc.), software (e.g., instructions executing on a processing device), or a combination thereof. It is to be appreciated that not all steps may be needed to perform the disclosure provided herein. Further, some of the steps may be performed simultaneously, concurrently, or in a different order than shown in FIG. 14, as will be understood by a person of ordinary skill in the art(s).

In 1401, sensor control device 102 and/or in vivo analyte sensor 104 may test the glucose levels of user 802 wearing sensor control device 102. Sensor control device 102 may receive one or more commands from data receiving device 120 to initiate data transfer, and in response, sensor control device 102 may be configured to wirelessly transmit stored analyte related data collected during a monitoring time period to data receiving device 120. Data receiving device 120 may receive and store the information. In some embodiments, sensor control device 102 or data receiving device 120 may transmit the information to trusted computer system 180.

In 1402, data receiving device 120 may generate and display an appropriate visualization of past readings of glucose data in a user interface. In one approach, trusted computer system 180 may generate the visualizations and transmit appropriate images to data receiving device 120 and cause data receiving device 120 to display the images in the user interface. The visualization may display past glucose readings in an appropriate format, such as a line graph, bar graph, or scatter graph. Suitable visualizations are displayed above in FIGS. 9A and 9C, with past readings 902 displaying the current and past readings of glucose data for a user as a line.

In 1403, data receiving device 120 and/or trusted computer system 180 may receive a user choice from user 802. The user choice may include meal choices, activity information, and other suitable user choices. In some examples, the available food choices may be provided by partner API 830 or by means of a single link into the partner's food choice portal. Once connected to the partner's food choice portal, user 802 may select a particular food item in a system managed by the partner (e.g., in the form of a separate application) or preferably, selected within the UI provided in data receiving device 120. A link may be established between data receiving device 120 and partner API 830 by using QR code 818 or physically tapping close to a NFC source (e.g. a modified small pad on each table in a restaurant). The content of the data communicated for each food choice may be a combination of fundamental parameters specific to that meal choice as well as the amount of the meal choice. Examples of fundamental parameters may include a mix of first-principles-based or clinical-practice-based parameters such as: (1) glycemic index, (2) glycemic load, (3) rate of glucose appearance profile in an averaged person with standard food bioavailability, (4) macronutrient composition (in percentage of carbohydrate, fat and protein), and (5) fiber content. A food choice portal may leverage a partner's food ordering system associated to that table and include a method of indicating a shared meal. Glycemic profile emulation subsystem 821 may use sharing information to determine a reduced glycemic impact of a particular meal choice. In another embodiment, the partner's food information may be provided via multiple links associated with each food item and/or food item combinations. In another embodiment, food choice information may be based on a reduced set of information such as the name of the meal without detailed fundamental meal parameter information. For example, the information can be obtained from a data linkage between the proposed system and wearable 106, where user 802 logs routine meals in the wearable's application. However, if the emulated glycemic profile is undesirable to the user, and the user decides to make selection changes, user 802 may cancel the recent meal selection via wearable 106 and potentially replace it with other choices.

In 1404, Glycemic profile emulation subsystem 821 may use the user choices and person-specific parameters to calculate a personalized glucose prediction. Person-specific parameters may be referred to herein as user-specific parameters. These are discussed in further detail below with reference to personalized parameters 1704. Glycemic profile emulation subsystem 821 may combine a recent glucose history, one or more parameters associated with a metabolic model, and information related to a user choice (such as food choice or exercise). As described below with reference to FIG. 17, Glycemic profile emulation subsystem 821 may employ a variety of machine learning models and user profiles to accurately predict future glucose levels based on the food choice referenced to user profiles. At first, Glycemic profile emulation subsystem 821 may rely on population-based parameters because any user-specific profile in final emulated glycemic profiles 1703 is not predictive enough. Population-based parameters may be parameters derived using data from a representative population. In contrast, person-specific parameters may be derived from data for a specific person. During that period, a sequence of user-interface modules may be provided to user 802 to remind the user the importance of providing as much information possible to get a more tailored experience. During this period, Glycemic profile emulation subsystem 821 may provide generalized predictions of their contemplated meal and activity choices without necessarily providing an exact what-if scenario of their near future glycemic profile.

In some embodiments, the machine learning model generates predicted glucose values and an associated prediction visualization (e.g., predicted glucose 1001B) in response to one or more inputs, such as user history (e.g., past readings 902), current user glucose information, and user input, such as various food options (e.g., provided via user input). Examples of output from the machine learning model include predicted glucose values, predicted minimum values, predicted maximum values, as well as the corresponding output visualizations that are bounded by these predicted values.

In 1405, visualization subsystem 824 may display an appropriate prediction for the future glucose levels across a future time range. The visualization data may be presented as a single trace (i.e., a line), a range, or using another suitable approach that allows the user to view future data relative to their latest glucose profile. This time range may be several minutes or several hours or other suitable time range. The time range may be, for example, at least 3 hours. The time range may be, for example, up to 5 hours. By way of further example, the time range may be 3 to 5 hours.

Example visualizations are provided above with reference to FIGS. 10A-B, 11A-1B, and 12A-6B and referred to as predicted glucose 1001A-1001F. For example, the prediction may incorporate TA180 metrics that provide a metric average over all of the logged meals over the past week. A post-meal TA180 is not the same as a TA180 metric calculated for a full twenty-four-hour period, which is likely greater. These two metrics are correlated to some degree and a linear correlation factor (or other functional fit) can be estimated based on either population data, per person, or both. The post-meal TA180 metric can be displayed in numerous ways. TA180 can be displayed as is or converted to an equivalent value for twenty-four hours by applying a correlation factor. The meal monitor application can provide a default twenty four-hour TA180 target value, and/or require/allow the user to set a value. A typical TA180 target value that is considered in good control for people with diabetes is about 30%, alternatively between about 25% to about 35%, alternatively between about 20% and about 40%.

In 1406, an optional step, data receiving device 120 may receive from user 802 an update to the user choice including portion size, sequence, activity details, etc. For example, user 802 may use sequence selector 1102 to edit the sequence of food items being consumed. For example, as displayed in FIG. 11B, user 802 may toggle whether a green salad is eaten before or after a tiramisu. For another example, as displayed in FIG. 12B, user 802 may edit the portion size of a food choice. Or for example, user 802 may edit the sequencing such that the user walks before or after the meal and/or increase the intensity or duration of an activity.

In 1407, based on the selection, Glycemic profile emulation subsystem 821 may calculate a predicted near future glucose impact based on the updated parameters. Glycemic profile emulation subsystem 821 may update particular parameters related to the food choices while keeping other parameters the same between iterations.

In 1408, visualization subsystem 824 may overlay the updated predicted glucose information in the visualization. This quick and easy interface for examining behavioral decisions makes the consequences of choices personally relatable in terms of their near future glycemic profile.

Seamless Integration with Partner Systems

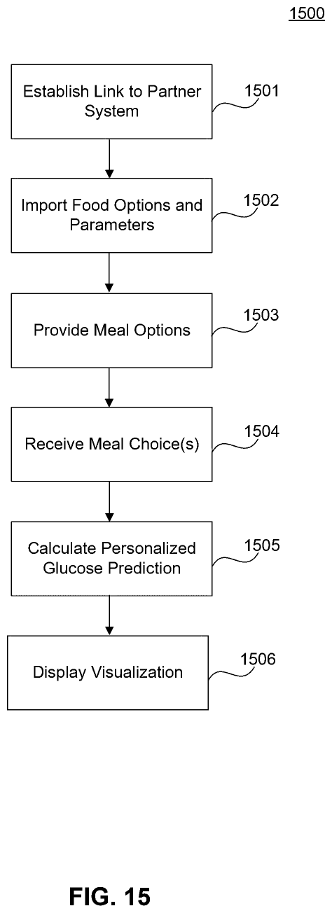

FIG. 15 is a flowchart illustrating a method 1500 of establishing a connection with a partner system to provide a personalized glucose prediction, according to some embodiments. Method 1500 can be performed by processing logic that can comprise hardware (e.g., circuitry, dedicated logic, programmable logic, microcode, etc.), software (e.g., instructions executing on a processing device), or a combination thereof. It is to be appreciated that not all steps may be needed to perform the disclosure provided herein. Further, some of the steps may be performed simultaneously, or in a different order than shown in FIG. 15, as will be understood by a person of ordinary skill in the art(s).

In 1501, partner integration subsystem 825 may establish a link to a partner system such as partner API 830. Such partner systems may include restaurants, diet programs, exercise programs, etc. In one embodiment, partner integration subsystem 825 may access menu options in response to user 802 scanning QR code 818 in a partner restaurant. Partner integration subsystem 825 may also access menu options via communications over NFC source 811.

In 1502, partner integration subsystem 825 may import food options and a communicable set of parameters for each menu choice by accessing partner API 830. For example, partner API 830 may provide an endpoint such as “get_items( )” that partner integration subsystem 825 may access to receive both the menu items selectable by user 802 and the parameters associated with each menu item used by Glycemic profile emulation subsystem 821 to predict a near future glucose value.

In 1503, data receiving device 120 may format and provide these meal options to user 802. For example, in response to a selection of an note visualization component 906 (as illustrated above in FIG. 9C), an appropriate page may display that allows user 802 to select a particular food option, adjust the quantity/portion, indicate sharing, etc.

In 1504, user 802 may select a particular menu choice. User 802 may also indicate the portion size of the item. Data receiving device 120 may provide differing methodologies for entering the meal information. For example, user 802 may select categories of fields such as the branded name of a food item as chosen by a partner restaurant (e.g., a blooming onion at the OUTBACK STEAKHOUSE®), generic meal name (e.g., whole fried onion), or a general category (e.g., fried appetizer). In another embodiment, user 802 may select a menu option in a partner system (e.g., in an ordering tool provided by the partner), and partner API 830 may communicate the user selection to data receiving device 120. By communicating the menu item to data receiving device 120, it obviates the need for user 802 to enter the menu selection in both the partner system and the data receiving device 120.

In 1505, Glycemic profile emulation subsystem 821 may calculate a personalized glucose prediction based on selected menu item. Glycemic profile emulation subsystem 821 may combine a recent glucose history, one or more parameters associated with a metabolic model, and information related to a user choice (such as food choice or exercise). The one or more parameters associated with a metabolic model may be specific to a user and may be referred to as user-specific parameters as discussed in further detail below with reference to personalized parameters 1704. Glycemic profile emulation subsystem 821 may employ a variety of machine learning models and user profiles to accurately predict future glucose levels based on the food choice referenced to user profiles.

In 1506, visualization subsystem 824 may display an appropriate prediction for the future glucose levels across a future time range. The visualization data may be presented as a single trace (i.e., a line), a range, or using another suitable approach that allows the user to view future data relative to their latest glucose profile. This time range may be several minutes or several hours or other suitable time range. Example visualizations are provided above with reference to FIGS. 10A-B, 11A-B, and 12A-B and referred to as predicted glucose 1001A-1001F.

### Connection to Wearable Device

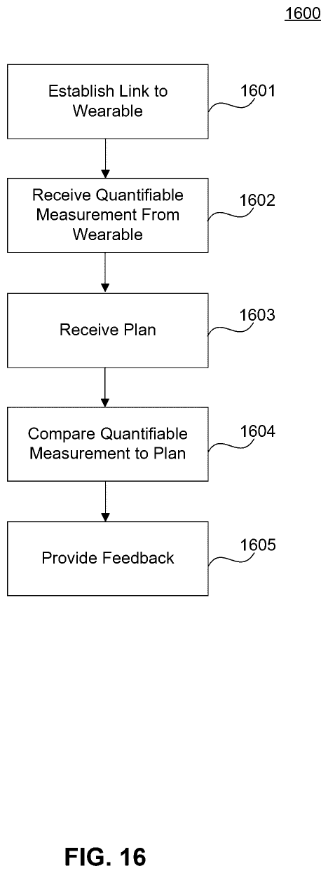

FIG. 16 is a flowchart illustrating a method of establishing a connection with a wearable to provide a personalized glucose prediction based on a physical activity. Method 1600 can be performed by processing logic that can comprise hardware (e.g., circuitry, dedicated logic, programmable logic, microcode, etc.), software (e.g., instructions executing on a processing device), or a combination thereof. It is to be appreciated that not all steps may be needed to perform the disclosure provided herein. Further, some of the steps may be performed simultaneously, or in a different order than shown in FIG. 16, as will be understood by a person of ordinary skill in the art(s).

In 1601, physical activity input subsystem 822 may establish a link to wearable 106. This may occur automatically or in response to user 802 commencing a synchronization process. This process may link and establish communications between wearable 106 and data receiving device 120, trusted computer system 180, and/or sensor control device 102.

In 1602, data receiving device 120 may receive a quantifiable measurement from wearable 106. The quantifiable measurement may be, e.g., a heart rate, a number of steps, respiration rate, and other data measured by wearable 106. In some embodiments, activity data may be entered by a user manually. In some embodiments, activity data may be obtained from a server or another app (e.g., APPLE HEALTH or HEALTH CONNECT APP).

In 1603, data receiving device 120 may retrieve a saved plan that includes one or more meals and/or exercise sessions as entered previously by user 802. For example, user 802 may have entered two future events—a particular meal choice and an exercise session—and viewed the impact on their future glucose levels. In some embodiments, user 802 may then save this information as a plan for the day, and data receiving device 120 may retrieve this stored plan at a later time.

In 1604, data receiving device 120 may compare the quantifiable measurement data to determine whether the retrieved saved plan is consistent with the actual behavior of user 802 as reflected in the quantifiable measurement data. To continue this example, data receiving device 120 may anticipate user 802 having an elevated heart rate at the time that the plan information specifies an exercise session.

In 1605, when the actual behavior diverges from the initial plan, data receiving device 120 may cause feedback to be sent to user 802, either via display 122 or wearable 106. The feedback may alert user 802 that their user action is inconsistent with the initial plan created in the interface. For example, if the user does not exercise at the time that the stored plan indicates an exercise session was supposed to occur, the user will not have an elevated heartrate. Data receiving device 120 may provide feedback to the user indicating that an exercise session was supposed to occur at the current time.

Learning and Adaption within the Glycemic Profile Emulation Subsystem

FIG. 17 is a block diagram of models and profiles used to generate an emulated glycemic profile, according to some embodiments. FIG. 17 may include pre-trained models 1701, personalized glycemic profiles 1702, emulated glycemic profiles 1703, personalized parameters 1704, and historical data 1705. In some embodiments, steps of FIG. 17 may be performed by one or more of Glycemic profile emulation subsystem 821, physical activity input subsystem 822, personalized glycemic profiles 823, and visualization subsystem 824, either singularly or in combination. In some embodiments, the output includes one or more visualization components for displaying the predicted glucose impact in combination with any one of the provided inputs, such as CGM sensor data, user medical history data, and other user inputs.

Pre-trained models 1701 may be models used by Glycemic profile emulation subsystem 821 to generate glycemic profiles that are used to predict near future glucose readings. In some embodiments, the pre-trained models 1701 may be implemented as a hybrid sensor-meal machine learning model trained, to receive as inputs, CGM sensor data, user medical history data, and user inputs related to meal choices. The hybrid sensor-meal machine learning model refers to a model trained to process data from disparate data sources, such as a CGM sensor, electronic health records (EHRs), and user-provided inputs. The machine learning model is configured to generate predicted glucose impacts based on the inputs and related visualizations for representing the inputs and outputs.

Pre-trained models 1701 may include both meal model inputs and exercise model inputs and model outputs. Meal model inputs may include glycemic index, glycemic load, food composition, food category labels, glucose appearance, profile features, pre-meal glucose, and other suitable values. Exercise model inputs may include steps per unit time, heart rates, an exercise mode, a duration, and other suitable values. The model outputs may be different classes of emulated glycemic profiles. Pre-trained models 1701 may include different meal models based on the one or more inputs—e.g., there may be a meal model that is tuned for different categories of users and/or meals. Categories of users may be organized based on common glycemic characteristics. Categories of meals may be organized based on meal type (such as breakfast, lunch, and dinner), restaurant, or food type. Similarly, there may be an exercise model that is tuned for different categories of users and/or exercises. Categories of users for exercise models may include organizing users based on common physical characteristics. Categories of exercises may include different exercise types (such as running, weightlifting). In an embodiment, near-future glucose pattern may be modeled/predicted by pre-processing training data and defining the model output as different classes of emulated glycemic profiles, with each class representing one typical glucose excursion. In this example, the modelling process is a supervised learning task, specifically a classification task. Therefore, some commonly used multiclass classification algorithms may be used for model training—e.g., multinomial Naive Bayes, K-nearest neighbors, decision trees, etc. In one approach, the model with the highest performance or predictive power may be used for future predictions. In some embodiments, the models described above may be generated via any known machine learning techniques—e.g., unsupervised learning, supervised learning, semi-supervised learning, re-informed learning, clustering, classification, regression, decision tree, neural networks, anomaly detection or any others.

Personalized glycemic profiles 1702 may include historical data 1705 and personalized parameters 1704. Personalized parameters 1704 may be personalized adjustment parameters, such as carbohydrate bioavailability and insulin sensitivity. Other personalized physiological parameters of interest may include time-to-peak gut absorption, glucose distribution volume, baseline endogenous glucose production rate, baseline insulin secretion, beta-cell function index, insulin-independent glucose utilization, gut hormone secretion, etc., depending on the complexity of the model structure. This allows maximum compatibility with meal and physical activity related information provided by partner systems band allows Glycemic profile emulation subsystem 821 to adapt to a particular user's metabolic condition over time. Personalized parameters 1704 may be updated over the time course when devices (e.g., sensor control device 102 and wearable 106) are worn. Historical data 1705 may be used to estimate/update the personalized parameters. An empirical model, such as the compartment model described above, with population a priori parameter estimates, may be adopted. Final emulated glycemic profiles 1703 may be fed by the historical data and posteriori individual parameter estimates.

Once generated, emulated glycemic profiles 1703 may be used for generating predictions of glucose readings for users. Glycemic profiles may be selected that have characteristics similar to user 802 and then utilized for generating predicted glucose readings based on inputs provided by 802. The inputs (e.g., meal, exercise, etc.) may be input to the selected glycemic profile(s) and the output may then result in the predicted glucose readings (or ranges) for the user based on those inputs.

Additional aspects of the machine learning model are discussed. The hybrid sensor-meal machine learning model is designed to generate nuanced visualizations for depicting potential relationships between user activity choices (e.g., lifestyle such as physical activity, health such as medical history and conditions, dietary such as meals) and glucose responses. The resulting hybrid sensor-meal visualization improves upon prior art user interfaces by bringing together data from disparate data sources (e.g., CGM sensor, third-party data sources, patient medical history, user input) and providing a user interface that depicts this information in a hybrid visualization format. The model's ability to provide individualized user activity insights (e.g., recommendations, coaching) helps users make informed decisions that can improve their overall well-being, particularly in managing conditions like diabetes.

In some embodiments, the hybrid sensor-meal visualization model is composed of multiple sub-models, some of which employ advanced machine learning techniques, while others can be based on rule-based logic. The hybrid sensor-meal visualization model can be trained using historical and population-based data on food consumption, user demographics, and post-prandial glucose measurements. Specifically, the training dataset can comprise labeled examples that link specific meals to corresponding glucose traces for individuals with comparable demographic and health histories, enabling the model to discern relationships between particular foods and their impact on glycemic responses.

In some embodiments, the hybrid sensor-meal visualization model includes a segmentation sub-model, which can employ unsupervised learning methodologies, such as clustering algorithms, to partition population data into multiple segments based on key features, including glucose response patterns, dietary preferences, and demographic characteristics. The segmentation sub-model can take as input one or more of food classification, post-prandial glucose traces related to the food classification, demographic data, and user preferences. The output of this model can be a segment assignment, categorizing the user into one of the predefined population segments.

The segmentation sub-model enables grouping individuals with similar physiological and behavioral traits and provide more accurate glucose impact predictions. The clustering algorithms used in the segmentation model are capable of identifying complex patterns and relationships within the data, such as correlations between specific foods and glucose responses that may vary significantly across different population groups.

In some embodiments, during an initialization period, the hybrid sensor-meal visualization model can relies predominantly on population-based parameters. During this initialization period, hybrid sensor-meal visualization model may lack sufficient user data to estimate a projected glycemic profile. During the initialization period, hybrid sensor-meal visualization model can output one or more user interface components for receiving user input including the user's medical history, user's glucose history, and other user parameters for generating personalized predictive glucose impacts. In some embodiments, during this period, the hybrid sensor-meal visualization model may provide generalized predictions based on contemplated meal and activity choices, and can update these predictions over time as additional user information is provided to the hybrid sensor-meal visualization model.

In some embodiments, the hybrid sensor-meal visualization model includes a glucose impact sub-model, which can utilize supervised learning to associate population segments with glucose data and predicted glucose impacts based on classified food items. This sub-model can be trained on data that links specific population segments to glucose impacts based on foods and known post-prandial glucose responses. The training process can involve utilizing labeled data that indicates which foods lead to desirable glycemic outcomes for individuals within each segment. The output of this sub-model can include the predicted glucose impact for a predetermined time period after a current time.

The glucose impact sub-model can use a combination of classification and regression techniques to predict the impact of various foods on an individual's glucose levels. By integrating demographic and health-related information, the glucose impact sub-model can refine its predictions as the user's health changes over time. For example, the user's glycemic response to certain foods can change over time as the user, for example, gains weight or loses weight. The supervised learning approach trains the sub-model's predicted glucose impacts based on empirical evidence derived from real-world data, enhancing the accuracy of predicted glucose impacts.

In some embodiments, the hybrid sensor-meal visualization model includes a meal intake sub-model, which can be trained to identify effects from different meal input information such as carbohydrate content, glycemic index, food composition, and order of food consumption. The meal intake sub-model output a dynamic profile of post meal glucose impacts based on the provided meal input information for the specific user based on the user's medical history and current glucose information.

In some embodiments, the hybrid sensor-meal visualization model includes a physical activity sub-module, which utilize supervised learning to associate types of physical activity with glucose impacts. The physical activity sub-module can be trained to identify patterns in the effect of physical activity and blood glucose levels, and can be further trained to personalize those patterns to specific users. The physical activity sub-module may receive, as inputs the mode of exercise and duration and intensity and can identify, for example, the impact of physical activity on lowering blood glucose up to a predetermined time period during and/or after the physical activity making the body more sensitive to insulin. As another example, the physical activity sub-module may determine that blood glucose for a particular user might also go up after certain intensive physical activity (e.g., heavy weightlifting, sprints and competitive sports), due to rising levels of stress hormones (such as adrenaline).

In some embodiments, the physical activity sub-module can model the effect of physical activity via different metrics such as intensity, duration and mode of exercise. Data of interest for the physical activity input module also include parameters that allow the sub-model to link the impact of physical activity to other measurable metrics that other connected wearables may measure (e.g. heart rate, steps, elevation change, etc.).

The hybrid sensor-meal visualization model can also incorporate a feedback loop within a visualization component that receives feedback on predicted glucose impacts. One example of this feedback may be provided by comparing the predicted glucose impact for a particular time period with an actual glucose impact provided by a connected CGM for that same period. This feedback can be used to further train the model, improving its predictive capabilities over time. By incorporating this feedback, the model becomes more adaptive and personalized to the glycemic response for each user, which will improve the accuracy of the predicted glucose impact over time.

The hybrid sensor-meal visualization model also supports the generation of dynamic user interface components for a user interface, where each component is configured to bring in data from different data sources, such as the user's CGM sensor, user medical record sources, and additional user input. These dynamic components provide real-time data visualization and personalized feedback based on the user's glucose levels and dietary inputs. For example, a user interface component may display a graph of glucose trends alongside recommended meals, enabling users to make informed dietary decisions. The integration of data from different sources allows for a more comprehensive and interactive user experience, helping users to visualize the direct impact of their food choices on their glucose levels.

The integration of machine learning-based and rule-based components facilitates a comprehensive and personalized approach to generating user activity (e.g., lifestyle such as physical activity, dietary such as meal choices) recommendations. By combining data-driven insights with user-defined rules, the hybrid sensor-meal visualization model can provide recommendations that are both scientifically grounded and tailored to individual preferences.

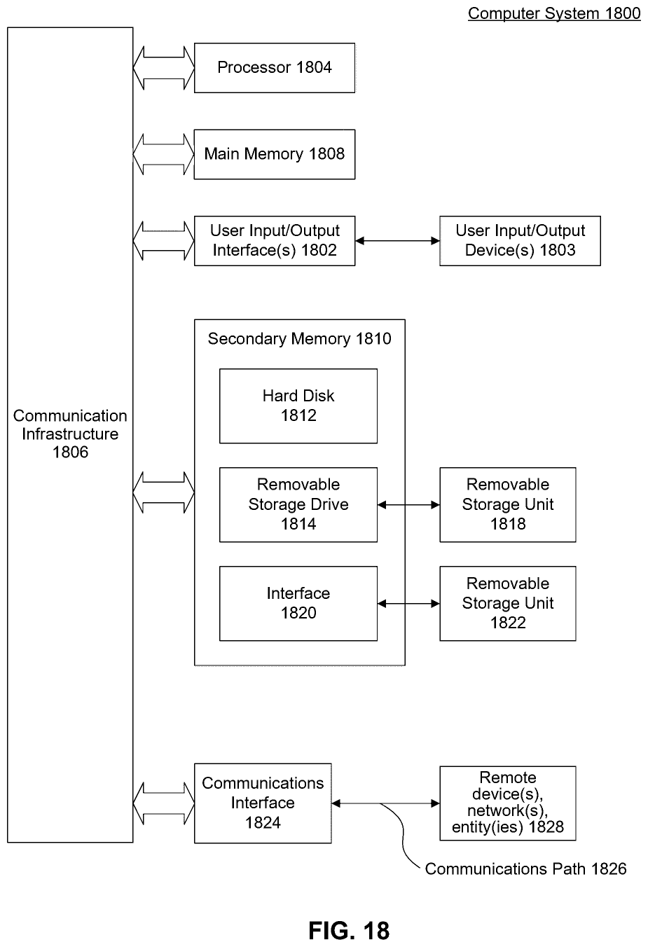

Various embodiments may be implemented, for example, using one or more well-known computer systems, such as computer system 1800 shown in FIG. 18. One or more computer systems 1800 may be used, for example, to implement any of the embodiments discussed herein, as well as combinations and sub-combinations thereof.

Computer system 1800 may include one or more processors (also called central processing units, or CPUs), such as a processor 1804. Processor 1804 may be connected to a communication infrastructure or bus 1806.

Computer system 1800 may also include user input/output device(s) 1808, such as monitors, keyboards, pointing devices, etc., which may communicate with communication infrastructure 1806 through user input/output interface(s) 1802.

One or more of processors 1804 may be a graphics processing unit (GPU). In an embodiment, a GPU may be a processor that is a specialized electronic circuit designed to process mathematically intensive applications. The GPU may have a parallel structure that is efficient for parallel processing of large blocks of data, such as mathematically intensive data common to computer graphics applications, images, videos, etc.

Computer system 1800 may also include a main or primary memory 1808, such as random access memory (RAM). Main memory 1808 may include one or more levels of cache. Main memory 1808 may have stored therein control logic (i.e., computer software) and/or data.

Computer system 1800 may also include one or more secondary storage devices or memory 1810. Secondary memory 1810 may include, for example, a hard disk drive 1812 and/or a removable storage device or drive 1814. Removable storage drive 1814 may be a floppy disk drive, a magnetic tape drive, a compact disk drive, an optical storage device, tape backup device, and/or any other storage device/drive.

Removable storage drive 1814 may interact with a removable storage unit 1818. Removable storage unit 1818 may include a computer usable or readable storage device having stored thereon computer software (control logic) and/or data. Removable storage unit 1818 may be a floppy disk, magnetic tape, compact disk, DVD, optical storage disk, and/any other computer data storage device. Removable storage drive 1814 may read from and/or write to removable storage unit 1818.

Secondary memory 1810 may include other means, devices, components, instrumentalities or other approaches for allowing computer programs and/or other instructions and/or data to be accessed by computer system 1800. Such means, devices, components, instrumentalities or other approaches may include, for example, a removable storage unit 1822 and an interface 1820. Examples of the removable storage unit 1822 and the interface 1820 may include a program cartridge and cartridge interface (such as that found in video game devices), a removable memory chip (such as an EPROM or PROM) and associated socket, a memory stick and USB port, a memory card and associated memory card slot, and/or any other removable storage unit and associated interface.

Computer system 1800 may further include a communication or network interface 1824. Communication interface 1824 may enable computer system 1800 to communicate and interact with any combination of external devices, external networks, external entities, etc. (individually and collectively referenced by reference number 1828). For example, communication interface 1824 may allow computer system 1800 to communicate with external or remote devices 1828 over communications path 1826, which may be wired and/or wireless (or a combination thereof), and which may include any combination of LANs, WANs, the Internet, etc. Control logic and/or data may be transmitted to and from computer system 1800 via communication path 1826.

Computer system 1800 may also be any of a personal digital assistant (PDA), desktop workstation, laptop or notebook computer, netbook, tablet, smart phone, smart watch or other wearable, appliance, part of the Internet-of-Things, and/or embedded system, to name a few non-limiting examples, or any combination thereof.

Computer system 1800 may be a client or server, accessing or hosting any applications and/or data through any delivery paradigm, including but not limited to remote or distributed cloud computing solutions; local or on-premises software (“on-premise” cloud-based solutions); “as a service” models (e.g., content as a service (CaaS), digital content as a service (DCaaS), software as a service (SaaS), managed software as a service (MSaaS), platform as a service (PaaS), desktop as a service (DaaS), framework as a service (FaaS), backend as a service (BaaS), mobile backend as a service (MBaaS), infrastructure as a service (IaaS), etc.); and/or a hybrid model including any combination of the foregoing examples or other services or delivery paradigms.

Any applicable data structures, file formats, and schemas in computer system 1800 may be derived from standards including but not limited to JavaScript Object Notation (JSON), Extensible Markup Language (XML), Yet Another Markup Language (YAML), Extensible Hypertext Markup Language (XHTML), Wireless Markup Language (WML), MessagePack, XML User Interface Language (XUL), or any other functionally similar representations alone or in combination. Alternatively, proprietary data structures, formats or schemas may be used, either exclusively or in combination with known or open standards.

In some embodiments, a tangible, non-transitory apparatus or article of manufacture comprising a tangible, non-transitory computer useable or readable medium having control logic (software) stored thereon may also be referred to herein as a computer program product or program storage device. This includes, but is not limited to, computer system 1800, main memory 1808, secondary memory 1810, and removable storage units 1818 and 1822, as well as tangible articles of manufacture embodying any combination of the foregoing. Such control logic, when executed by one or more data processing devices (such as computer system 1800), may cause such data processing devices to operate as described herein.

Based on the teachings contained in this disclosure, it will be apparent to persons skilled in the relevant art(s) how to make and use embodiments of this disclosure using data processing devices, computer systems and/or computer architectures other than that shown in FIG. 18. In particular, embodiments can operate with software, hardware, and/or operating system implementations other than those described herein.

It is to be appreciated that the Detailed Description section, and not any other section, is intended to be used to interpret the claims. Other sections can set forth one or more but not all exemplary embodiments as contemplated by the inventor(s), and thus, are not intended to limit this disclosure or the appended claims in any way.

While this disclosure describes exemplary embodiments for exemplary fields and applications, it should be understood that the disclosure is not limited thereto. Other embodiments and modifications thereto are possible, and are within the scope and spirit of this disclosure. For example, and without limiting the generality of this paragraph, embodiments are not limited to the software, hardware, firmware, and/or entities illustrated in the figures and/or described herein. Further, embodiments (whether or not explicitly described herein) have significant utility to fields and applications beyond the examples described herein.

Embodiments have been described herein with the aid of functional building blocks illustrating the implementation of specified functions and relationships thereof. The boundaries of these functional building blocks have been arbitrarily defined herein for the convenience of the description. Alternate boundaries can be defined as long as the specified functions and relationships (or equivalents thereof) are appropriately performed. Also, alternative embodiments can perform functional blocks, steps, operations, methods, etc. using orderings different than those described herein.

References herein to “one embodiment,” “an embodiment,” “an example embodiment,” or similar phrases, indicate that the embodiment described can include a particular feature, structure, or characteristic, but every embodiment can not necessarily include the particular feature, structure, or characteristic. Moreover, such phrases are not necessarily referring to the same embodiment. Further, when a particular feature, structure, or characteristic is described in connection with an embodiment, it would be within the knowledge of persons skilled in the relevant art(s) to incorporate such feature, structure, or characteristic into other embodiments whether or not explicitly mentioned or described herein. Additionally, some embodiments can be described using the expression “coupled” and “connected” along with their derivatives. These terms are not necessarily intended as synonyms for each other. For example, some embodiments can be described using the terms “connected” and/or “coupled” to indicate that two or more elements are in direct physical or electrical contact with each other. The term “coupled,” however, can also mean that two or more elements are not in direct contact with each other, but yet still co-operate or interact with each other.

The breadth and scope of this disclosure should not be limited by any of the above-described exemplary embodiments, but should be defined only in accordance with the following claims and their equivalents.

The present invention is also described in accordance with the following clauses: Clause 1. receiving, at a data receiving device, a glucose reading from an in vivo glucose sensor in communication with the data receiving device, wherein the glucose reading comprises a current glucose value at a current time and glucose data for a predetermined time period;
generating a visualization of the glucose reading on a mobile application running on the data receiving device wherein the visualization comprises a first visualization component for displaying the current glucose value and a second visualization component for displaying the glucose data for the predetermined time period;
receiving a user choice via a graphical user interface of the mobile application;
calculating a personalized glucose prediction based on the user choice, user-specific parameters, and the glucose reading, wherein the personalized glucose prediction is for a predetermined future time period subsequent to the current time; and
updating the second visualization component to display the personalized glucose prediction as an extension of the glucose data.

Clause 2. The method of clause 1, the calculating further comprising:
retrieving one or more parameters associated with the user choice, wherein the one or more parameters comprise a glycemic index, a glycemic load, a rate of glucose appearance profile in an averaged person with standard food bioavailability, a macronutrient composition, a sequence, and/or a fiber content.

Clause 3. The method of clause 2, further comprising:
retrieving the one or more parameters associated with the user choice via a call to an application programming interface provided by a partner system.

Clause 4. The method of any preceding clause, wherein the personalized analyte prediction displays as a single trace line and is presented graphically proximate to the visualization of the analyte readings.

Clause 5. The method of any preceding clause, wherein the personalized analyte prediction comprises a range of values.

Clause 6. The method of any preceding clause, wherein the user choice is a first user choice, further comprising:
receiving a second user choice and a sequencing that indicates a temporal order of the second user choice and the user choice via the graphical user interface of the mobile application; and
calculating an updated personalized analyte prediction based on the user choice, the second user choice, the sequencing, the user-specific parameters, and the analyte readings.

Clause 7. The method of any preceding clause, further comprising:
receiving a portion reduction for the user choice via the graphical user interface of the mobile application; and
calculating an updated personalized analyte prediction based on the portion reduction and the personalized analyte prediction.

Clause 8. The method of any preceding clause, wherein the recent time is a current time.

Clause 9. The method of any preceding clause, wherein the personalized analyte prediction spans a future time range of, at least 3 hours and/or up to 5 hours, preferably the time range is 3 to 5 hours.

Clause 10. The method of any preceding clause, further comprising:
receiving a physical activity via the graphical user interface of the mobile application; and
calculating the personalized analyte prediction based on the physical activity, the user-specific parameters, and the analyte readings.

Clause 11. The method of clause 10, the calculating further comprising:
retrieving one or more parameters associated with the physical activity, wherein the one or more parameters associated with the physical activity comprise intensity, duration of exercise, mode of exercise, endogenous glucose production, stress hormone levels, insulin sensitivity, a sequence of exercise, and/or demographics information.

Clause 12. The method of any preceding clause, further comprising:
recording the user choice as an initial plan;
receiving a quantifiable measurement from a connected wearable device; and
providing feedback on whether the quantifiable measurement is consistent with the initial plan.

Clause 13. The method of any preceding clause, further comprising:
receiving a second user choice via a real-time user input component of the graphical user interface of the mobile application, wherein the second user choice is configured to adjust one or more values of the user choice;
updating the personalized glucose prediction based the second user choice; and
updating, in real-time, the second visualization component to display the personalized glucose prediction as an extension of the glucose data.

Clause 14. The method of any preceding clause, further comprising:
retrieving a pre-trained model based on the user-specific parameters;
generating a personal glycemic profile using the pre-trained model; and
calculating the personalize analyte prediction using the personalized glycemic profile.

Clause 15. A system comprising:
a memory;
a processor coupled to the memory and configured to:
receive a glucose reading from an in vivo glucose sensor in communication with the system, wherein the glucose reading comprises a current glucose value at a current time and glucose data for a predetermined time period;
generate a visualization of the glucose reading on a mobile application running on the system wherein the visualization comprises a first visualization component for displaying the current glucose value and a second visualization component for displaying the glucose data for the predetermined time period;
receive a user choice via a graphical user interface of the mobile application;
calculate a personalized glucose prediction based on the user choice, user-specific parameters, and the glucose reading, wherein the personalized glucose prediction is for a predetermined future time period subsequent to the current time; and
update the second visualization component to display the personalized glucose prediction as an extension of the glucose data.

Clause 16. The system of clause 15, wherein to calculate the personalized analyte prediction, the processor is further configured to:
retrieve one or more parameters associated with the user choice, wherein the one or more parameters associated with the user choice comprise a glycemic index, a glycemic load, a rate of glucose appearance profile in an averaged person with standard food bioavailability, a macronutrient composition, a sequence, and/or a fiber content.

Clause 17. The system of clause 15, the processor further configured to:
receive a physical activity via the graphical user interface of the mobile application; and
calculate the personalized analyte prediction based on the physical activity, the user-specific parameters, and the analyte readings.

Clause 18. The system of clause 17, the calculating further comprising:
retrieve one or more parameters associated with the physical activity, wherein the one or more parameters associated with the physical activity comprise intensity, duration of exercise, mode of exercise, endogenous glucose production, stress hormone levels, insulin sensitivity, a sequence of exercise, and/or demographics information.

Clause 19. The system of clause 15, the processor further configured to:
receive a second user choice via a real-time user input component of the graphical user interface of the mobile application, wherein the second user choice is configured to adjust one or more values of the user choice;
update the personalized glucose prediction based the second user choice; and
update, in real-time, the second visualization component to display the personalized glucose prediction as an extension of the glucose data.

Clause 20. A non-transitory computer-readable device having instructions stored thereon that, when executed by a computing device, causes the computing device to perform operations comprising:
receiving, at a data receiving device, a glucose reading from an in vivo glucose sensor in communication with the data receiving device, wherein the glucose reading comprises a current glucose value at a current time and glucose data for a predetermined time period;
generating a visualization of the glucose reading on a mobile application running on the data receiving device wherein the visualization comprises a first visualization component for displaying the current glucose value and a second visualization component for displaying the glucose data for the predetermined time period;
receiving a user choice via a graphical user interface of the mobile application; calculating a personalized glucose prediction based on the user choice, user-specific parameters, and the glucose reading, wherein the personalized glucose prediction is for a predetermined future time period subsequent to the current time; and
updating the second visualization component to display the personalized glucose prediction as an extension of the glucose data.

What is claimed is:

1. A method, comprising:
receiving, at a data receiving device, a glucose reading from an in vivo glucose sensor in communication with the data receiving device, wherein the glucose reading comprises a recent glucose value at a recent time and glucose data for a predetermined time period;
generating a visualization of the glucose reading on a mobile application running on the data receiving device wherein the visualization comprises a first visualization component for displaying the recent glucose value and a second visualization component for displaying the glucose data for the predetermined time period;
receiving a user choice via a graphical user interface of the mobile application;
calculating a personalized glucose prediction based on the user choice, user-specific parameters, and the glucose reading, wherein the personalized glucose prediction is for a predetermined future time period subsequent to the recent time; and
updating the second visualization component to display the personalized glucose prediction as an extension of the glucose data.

2. The method of claim 1, the calculating further comprising:
retrieving one or more parameters associated with the user choice, wherein the one or more parameters comprise a glycemic index, a glycemic load, a rate of glucose appearance profile in an averaged person with standard food bioavailability, a macronutrient composition, a sequence, and/or a fiber content.

3. The method of claim 2, further comprising:
retrieving the one or more parameters associated with the user choice via a call to an application programming interface provided by a partner system.

4. The method of claim 1, wherein the personalized glucose prediction displays as a single trace line and is presented graphically proximate to the visualization of the glucose readings.

5. The method of claim 1, wherein the personalized glucose prediction comprises a range of values.

6. The method of claim 1, wherein the user choice is a first user choice, further comprising:
receiving a second user choice and a sequencing that indicates a temporal order of the second user choice and the user choice via the graphical user interface of the mobile application; and
calculating an updated personalized glucose prediction based on the user choice, the second user choice, the sequencing, the user-specific parameters, and the glucose readings.

7. The method of claim 1, further comprising:
receiving a portion reduction for the user choice via the graphical user interface of the mobile application; and
calculating an updated personalized glucose prediction based on the portion reduction and the personalized glucose prediction.

8. The method of claim 1, wherein the recent time is a current time.

9. The method of claim 1, wherein the personalized glucose prediction spans a future time range of at least 3 hours and/or up to 5 hours.

10. The method of claim 1, further comprising:
receiving a physical activity via the graphical user interface of the mobile application; and
calculating the personalized glucose prediction based on the physical activity, the user-specific parameters, and the glucose readings.

11. The method of claim 10, the calculating further comprising:
retrieving one or more parameters associated with the physical activity, wherein the one or more parameters associated with the physical activity comprise intensity, duration of exercise, mode of exercise, endogenous glucose production, stress hormone levels, insulin sensitivity, a sequence of exercise, and/or demographics information.

12. The method of claim 1, further comprising:
recording the user choice as an initial plan;
receiving a quantifiable measurement from a connected wearable device; and
providing feedback on whether the quantifiable measurement is consistent with the initial plan.

13. The method of claim 1, further comprising:
receiving a second user choice via a real-time user input component of the graphical user interface of the mobile application, wherein the second user choice is configured to adjust one or more values of the user choice;
updating the personalized glucose prediction based the second user choice; and
updating, in real-time, the second visualization component to display the personalized glucose prediction as an extension of the glucose data.

14. The method of claim 1, the calculating the personalized glucose prediction further comprising:
retrieving a pre-trained model based on the user-specific parameters;
generating a personal glycemic profile using the pre-trained model; and
calculating the personalize glucose prediction using the personalized glycemic profile.

15. A system comprising:
a memory;
a processor coupled to the memory and configured to:
receive a glucose reading from an in vivo glucose sensor in communication with the system, wherein the glucose reading comprises a recent glucose value at a recent time and glucose data for a predetermined time period;
generate a visualization of the glucose reading on a mobile application running on the system wherein the visualization comprises a first visualization component for displaying the recent glucose value and a second visualization component for displaying the glucose data for the predetermined time period;
receive a user choice via a graphical user interface of the mobile application;
calculate a personalized glucose prediction based on the user choice, user-specific parameters, and the glucose reading, wherein the personalized glucose prediction is for a predetermined future time period subsequent to the recent time; and
update the second visualization component to display the personalized glucose prediction as an extension of the glucose data.

16. The system of claim 15, wherein to calculate the personalized glucose prediction, the processor is further configured to:
retrieve one or more parameters associated with the user choice, wherein the one or more parameters associated with the user choice comprise a glycemic index, a glycemic load, a rate of glucose appearance profile in an averaged person with standard food bioavailability, a macronutrient composition, a sequence, and/or a fiber content.

17. The system of claim 15, the processor further configured to:
receive a physical activity via the graphical user interface of the mobile application; and
calculate the personalized glucose prediction based on the physical activity, the user-specific parameters, and the glucose readings.

18. The system of claim 17, the calculating further comprising:
retrieve one or more parameters associated with the physical activity, wherein the one or more parameters associated with the physical activity comprise intensity, duration of exercise, mode of exercise, endogenous glucose production, stress hormone levels, insulin sensitivity, a sequence of exercise, and/or demographics information.

19. The system of claim 15, the processor further configured to:
receive a second user choice via a real-time user input component of the graphical user interface of the mobile application, wherein the second user choice is configured to adjust one or more values of the user choice;
update the personalized glucose prediction based the second user choice; and
update, in real-time, the second visualization component to display the personalized glucose prediction as an extension of the glucose data.

20. A non-transitory computer-readable device having instructions stored thereon that, when executed by a computing device, causes the computing device to perform operations comprising:
receiving, at a data receiving device, a glucose reading from an in vivo glucose sensor in communication with the data receiving device, wherein the glucose reading comprises a recent glucose value at a recent time and glucose data for a predetermined time period;
generating a visualization of the glucose reading on a mobile application running on the data receiving device wherein the visualization comprises a first visualization component for displaying the recent glucose value and a second visualization component for displaying the glucose data for the predetermined time period;
receiving a user choice via a graphical user interface of the mobile application;
calculating a personalized glucose prediction based on the user choice, user-specific parameters, and the glucose reading, wherein the personalized glucose prediction is for a predetermined future time period subsequent to the recent time; and
updating the second visualization component to display the personalized glucose prediction as an extension of the glucose data.
